(19) United States
(12) Patent Application Publication
(10) Pub. No.: US 2025/0268870 A1
(43) Pub. Date: Aug. 28, 2025

(54) SMALL MOLECULE INDUCERS OF CARDIOMYOCYTES TO IMPROVE CARDIAC STRUCTURE AND FUNCTION

(71) Applicant: UNIVERSITY OF MARYLAND, BALTIMORE, Baltimore, MD (US)

(72) Inventors: Charles C. HONG, Baltimore, MD (US); Young Wook CHUN, Woodstock, MD (US); Matthew MIYAMOTO, Baltimore, MD (US)

(21) Appl. No.: 18/857,231

(22) PCT Filed: Apr. 17, 2023
(86) PCT No.: PCT/US2023/018866

§ 371 Date: Oct. 16, 2024

Related U.S. Application Data
(60) Provisional application No. 63/332,004, filed on Apr. 18, 2022.

(51) Int. Cl. A61K 31/433; A61K 31/196; A61K 31/409; A61K 31/505; A61P 9/04

(52) U.S. Cl. CPC A61K 31/433; A61K 31/196; A61K 31/409; A61K 31/505; A61P 9/04

(57) ABSTRACT

Here, we describe an infant with congenital dilated cardiomyopathy (cDCM) whose impaired cardiac function and disrupted sarcomere and mitochondria structures were modeled using induced pluripotent stem cells (iPSCs). The causal gene encodes the centrosomal protein rotatin (RTTN), representing the first time a centrosome defect has been found to cause nonsyndromic dilated cardiomyopathy (DCM) in humans. Genetic knockdowns in zebrafish and Drosophila confirmed an evolutionarily conserved requirement of RTTN for cardiac structure and function. The small molecule, C19, restored initiation of the perinuclear MTOC, and significantly improved the structure and function of cardiomyocytes. In summary, this study provides a new therapeutic strategy for infantile or congenital DCM.

### CROSS-REFERENCE TO RELATED APPLICATIONS

This application is a 371 National Stage application of PCT Application No. PCT/US23/18866, filed Apr. 17, 2023, which claims the benefit of U.S. Provisional Application No. 63/332,004, filed 18 Apr. 2022. The entire contents of each of these applications is hereby incorporated by reference as if fully set forth herein.

### GOVERNMENT FUNDING SUPPORT

This invention was made with government support under grant no. HL135129, awarded by the National Institutes of Health. The government has certain rights in the invention.

### REFERENCE TO SEQUENCE LISTING

This application contains a Sequence Listing which has been submitted electronically in .XML format and is hereby incorporated by reference in its entirety for the above-identified application. Said .XML file, created on Jul. 5, 2023, is named “15024-381PC0.xml” and is 61,432 bytes in size. The sequence listing contained in the .XML file is part of the specification and is hereby incorporated by reference herein in its entirety.

### BACKGROUND

### 1. Field of the Invention

The invention relates to the general field of medicine, and in particular to small molecule inducers of cardiomyocyte maturation or cell cycle arrest for use in heart disease. Small Molecule Inducers such as C19 have been found to restore cardiac structure, function, and metabolism, useful in treating congenital and adult diseases and conditions of the heart. For example, the invention is useful for congenital/infantile dilated cardiomyopathy (cDCM/iDCM) and centrosome reorganization in infantile heart failure. The administration of small molecule C 19 can be sufficient to reverse cardiomyopathy (including improving contractility) characterized by myocyte immaturity and reduce the burden of transplants on patients and hospitals.

### 2. Background of the Invention

Infantile dilated cardiomyopathy (iDCM) is a rare condition of multiple etiologies which causes significant morbidity and mortality. Of the studied infantile cardiomyopathies, dilated cardiomyopathy is the most prevalent, responsible for approximately 50% of infantile/pediatric cardiomyopathy cases. Such cases are particularly difficult to manage, and many end up requiring heart transplant as the final management approach. Given the lack of donor hearts available, particularly in the infant demographic, there is thus a significant need to better understand the etiology of iDCM for development of tools for identification and targeted approaches for effective therapeutics.

Previous studies have suggested that 27-54% of iDCM cases have a genetic component. Common mutations in genes related to sarcomere structure, such as MYH7, MYBPC3 and TNNT2, nuclear envelope, such as LMNA, and cytoskeleton, such as DES and DMD, have been described previously. Increased widespread genetic testing abilities have begun to help identify novel classes of genes suspected to be responsible for iDCM, greatly expanding our understanding of the etiology of this condition. See Table 1, below.

TABLE 1
Gene List Associated with Dilated Cardiomyopathy.

| | | | | |
|---|---|---|---|---|
| ABCC6 | DYSF | HAND1 | MT-CYB | MT-TLTL21 |
| ABCC9 | EEF1A2 | HCN4 | MT-ND1 | MT-TM |
| ACTA1 | EMD | ILK | MT-ND2 | MT-TN |
| ACTC1 | EPG5 | JPH2 | MT-ND3 | MT-TP |
| ACTN2 | ETFA | JUP | MT-ND4 | MT-TQ |
| ALMS1 | ETFB | KLHL24 | MT-ND4L | MT-TR |
| ALPK3 | ETFDH | LAMP2 | MT-ND5 | MT-TS1 |
| APOA1 | FBXO32 | LDB3 | MT-ND6 | MT-TS2 |
| BAG3 | FHOD3 | LEMD2 | MT-RNR1 | MT-TT |
| CASZ1 | FKTN | LMNA | MT-RNR2 | MT-TV |
| CHRM2 | FLNC | LMOD2 | MT-TA | MT-TW |
| DES | FOXD4 | LRRC10 | MT-TD | MT-TY |
| DMD | GATA4 | MLYCD | MT-TE | MYBPC3 |
| DOLK | GATA5 | MT-ATP6 | MT-TF | MYBPHL |
| DPM3 | GATC | MT-ATP8 | MT-TG | MYH6 |
| DSC3 | GBE1 | MT-CO1 | MT-TH | MYH7 |
| DSG2 | GLB1 | MT-CO2 | MT-TI | MYL3 |
| DSP | GSK3B | MT-CO3 | MT-TK | NKX2-5 |
| NRAP | PRDM16 | SPEG | TNNC1 | TTR |
| PCCA | QRSL1 | TAB2 | TNNI3 | VCL |
| PCCB | RAF1 | TAZ | TNNI3K | VPS13A |
| PKP2 | RBCK1 | TBX20 | TNNT2 | |
| PLEKKHM2 | RBM20 | TBX5 | TOR1AIP1 | |
| PLN | RMND1 | TCAP | TPM1 | |
| PPCS | SCN5A | TMEM43 | TTN | |

During cardiomyocyte (CM) maturation, the centrosome, which functions as a microtubule organizing center (MTOC) in CMs, undergoes dramatic structural change during which its components reorganize from being localized at the centriole to the nuclear envelope. This developmentally programmed process, referred to as centrosome reduction, has been previously associated with cell cycle exit. However, understanding how this process influences CM cell biology, and whether its disruption results in human cardiac disease, remains unknown.

### SUMMARY OF THE INVENTION

Therefore, there is a need in the art for methods for treating certain congenital and adult heart conditions. In this study, this phenomenon is investigated in an infant with a rare case of infantile dilated cardiomyopathy (iDCM) who presented with left ventricular ejection fraction of 18% and disrupted sarcomere and mitochondria structure.

In particular embodiments, the present invention relates to a method of treating a cardiomyopathy in a subject in need thereof, comprising administering the subject a Hippo pathway activator compound. Preferably, the Hippo pathway activator compound is selected from Verteporfin, Compound 13, DAY10583, CA3, SBI-115 and C19, and most preferably is C19.

In particular embodiments, the subject is a human or a human infant.

In some embodiments, administration to the subject is by intravenous infusion, intravenous injection, subcutaneous injection, or orally.

In some embodiments, the subject suffers from infantile dilated cardiomyopathy, acquired dilated cardiomyopathy, a left ventricular non-compaction cardiomyopathy, a pediatric dilated cardiomyopathy, or heart failure secondary to congenital heart disease.

### BRIEF SUMMARY OF THE DRAWINGS

Certain embodiments are illustrated by way of example, and not by way of limitation, in the figures of the accompanying drawings.

FIG. 1 is a workflow diagram for whole-exome sequencing analysis to identify the causal gene.

FIG. 2A, FIG. 2B, FIG. 2C, FIG. 2D, and FIG. 2E are a set of photographs of two independent iPSC cell lines derived from the cDCM (iDCM) patient. Each line had embryonic stem cell-like morphology and expressed the pluripotent stemness markers alkaline phosphatase (ALP), NANOG, Oct4, and SSEA4. Scale bars=10 μm.

FIG. 3 shows the karyotypes of the cDCM-iPSC which appear normal.

FIG. 4A and FIG. 4B are a set of graphs showing confirmation by flow cytometry that the iPSC-CMs generated by directed differentiation were positive for troponin.

FIG. 5 is a graph showing that more than 90% of control, cDCM, KO, and GC lines were α-actinin positive at 30 days after cardiac differentiation. Scale bar=10 μm.

FIG. 6A provides the general strategy for generation of RTTN KO and GC iPSC-CMs. The region corresponding to p.G1321 on exon 29 was targeted for KO in control iPSCs by nonhomologous end-joining (NHEJ), and the region containing the p.G1321D mutation in cDCM iPSC edited by homology-directed repair (HDR).

FIG. 6B presents a single-guide RNA (sgRNA) (SEQ ID NO:7) targeting the PAM site (green) in RTTN exon 29.

FIG. 6C shows the four independent KO lines that were confirmed by Sanger sequencing. Dotted lines indicate deletions and red letters indicate insertions. FIG. 6C provides the following sequences: KO-1 (SEQ ID NOS: 8 and 46); KO-2 (SEQ ID NOS: 9 and 47); KO-3 (SEQ ID NOS: 10 and 48); and KO-4 (SEQ ID NOS: 11 and 49).

FIG. 6D provides sgRNA for genome correction in cDCM iPSCs targets the PAM site (green) near the region on exon 29 that corresponds to the p.G1321D mutation (violet). The repair templates contains the reference sequence at G4018 (violet), corresponding to p.G1321. The repair template also contained a “beacon” (red), which does not change the amino acid sequence, to confirm gene repair. RTTN at exon 29 (SEQ ID NO:12) and the repair template (SEQ ID NO:13) are shown.

FIG. 6E presents information on a total of nine gene-corrected iPSC lines that were generated and confirmed by Sanger sequencing. The CRISPR-Cas9-mediated generation of RTTN KO and GC lines was able to confirm RTTN as the causal gene.

FIG. 7A and FIG. 7B are transmission electron micrographs of heart tissue. FIG. 7A shows a normal heart with well-organized myofilaments, distinct Z-lines (see Z arrowheads) and mitochondria (“Mito”). FIG. 7B shows the heart of an iDCM patient with severely disorganized myofibrils with indistinct Z-lines and dysmorphic mitochondria (Mito) without appreciable cristae. Scale bar=1 μm.

FIGS. 7C and 7D present iPSC-CM from a healthy control (FIG. 7C) exhibiting organized myofilaments with distinct Z-lines (Z; arrowheads) and iPSC-CM from an iDCM patient (FIG. 7D) exhibiting disorganized myofibrils without distinct Z-lines (Z). Scale bar=0.5 μm.

FIGS. 7E and 7F present representative immunofluorescence images of α-actinin staining of control (FIG. 7E) and iDCM (FIG. 7F) cardiomyocytes (CMs). Scale bar=10 μm.

FIG. 8A, FIG. 8B, FIG. 8C, FIG. 8D, FIG. 8E, and FIG. 8F are a set of photomicrographs showing that the control iPSC-CMs (top) exhibited organized sarcomere structures, whereas the sarcomeres in the cDCM iPSC-CMs (bottom) exhibited disarray. Cardiac troponin T (green), α-actinin (orange). Scale bars=10 μm.

FIG. 9A and FIG. 9B are a set of photomicrographs showing that the control iPSC-CMs exhibited a network of elongated mitochondria (MitoTracker, green), whereas cDCM iPSC-CMs displayed globular, punctate mitochondria.

FIG. 10 presents data comparing control-CMs derived from 2 independent healthy control iPSC lines (Control-1, -2) and iDCM-CMs from 2 independent iPSC lines (iDCM-1, -2).

FIG. 11A and FIG. 11B are transmission electron micrographs of iPSC-CMs, focused on mitochondria. FIG. 11A shows the mitochondria (arrowheads) of control iPSC-CMs, which are easily distinguishable and have distinct cristae. FIG. 11B shows the mitochondria (arrowheads) from iDCM-CMs, which appear larger and swollen, without clear cristae. Scale bar=0.5 μm.

FIG. 12 presents data from fluorescence-activated cell sorting analysis of iDCM-CMs.

FIG. 13 provides sequences for proband and mother (DNA; SEQ ID NO:1), proband and mother (amino acid; SEQ ID NO:2), father (DNA; SEQ ID NO:3), father (amino acid; SEQ ID NO:4), proband and father (DNA; SEQ ID NO:5), proband and father (amino acid; SEQ ID NO:6), mother (DNA; SEQ ID NO:44), and mother (amino acid; SEQ ID NO:45).

FIG. 14 is a set of representative immunofluorescence images showing that KO and GC iPSC lines were Oct4- and TRA-60-positive.

FIG. 15A and FIG. 15B present transmission electron micrographs of RTTN knockout (KO) CMs and gene-corrected (GC) CMs, respectively. Scale bar=0.5 μm.

FIG. 16A and FIG. 16B show mitochondria visualized by MitoTracker™ (green) and myofilaments visualized by alpha-actinin staining (red) in RTTN KO-CMs (FIG. 16A) and RTTN GC-CMs (FIG. 16B), respectively. Scale bar=20 μm.

FIG. 17 is a set of photomicrographs showing that RTTN KO iPSC-CMs exhibited disorganized sarcomeres (red, α-actinin staining of Z-line), whereas RTTN GC iPSC-CMs exhibited grossly normal sarcomere structure.

FIG. 18 presents cell shortening data for the indicated CMs. Center line=median; whiskers=1.5IQR.

FIG. 19A and FIG. 19A present data from FACS analysis of TMRM staining in KO and GC CMs.

FIG. 20A and FIG. 20B present FACS analysis indicating that control and cDCM iPSC-neurons have quantitatively similar mitochondrial function.

FIG. 21A shows that control and cDCM iPSC-derived astrocytes exhibited no differences in gross morphology, mitochondrial morphology (MitoTracker, green), or mitochondrial function (TMRM, red). Scale bars=50 μm.

FIG. 21B shows that control and cDCM iPSC-derived neurons exhibited no differences in gross morphology, mitochondrial morphology (MitoTracker, green), or mitochondrial function (TMRM, red). Scale bars=50 μm.

FIG. 22 presents images of embryos at 48 hours post fertilization (hpf): (i) uninjected embryo; (ii) embryo injected with translational blocking morpholino (MO); embryos injected with CRISPRi #1 (iii) and #2 (iv). The corresponding enlarged images of the heart are shown on the right with the arrowhead at the outline of the heart.

FIG. 23A shows the incidence of pericardial edema with impaired tail circulation in 48-hpf embryos. FIG. 23B shows abnormal development morphologies in mice. FIG. 23C show microcephaly in fish.

FIG. 23D, FIG. 23E, and FIG. 23F show that there were no significant increases in the occurrence of hydrocephaly, spinal curvature, or short body axis in the fish.

FIG. 23G and FIG. 23H show successful knockdown of RTTN in zebrafish embryos via RT-qPCR.

FIG. 23I presents micrographs of tissue stained with F-Actin in knockdown embryos, showing regions of sarcomere malformation (both morpholino and CRISPRi). FIG. 23J is a set of micrographs wherein a mitochondrial GFP tag in zebrafish suggested disruption of mitochondria in RTTN morphants.

FIG. 24A, FIG. 24B, and FIG. 24C present immunofluorescence images of actin, actinin, and acetylated (Ac) alpha-tubulin, respectively, in adult hearts (segment A4) from control (w1118), ana3^{+/-} and ana3^{-/-} flies. Scale bar=50 μm. These data show that knockdown of ana3 in Drosophila leads to impaired sarcomere and microtubule formation.

FIG. 25A and FIG. 25B are graphs showing numbers of embryos with heart malformations and abnormal heart looping, respectively.

FIG. 25C, FIG. 25D, FIG. 25E, and FIG. 25E show results for microcephaly, hydrocephaly, spinal curvature, and short body axis, respectively, as indicated. These trends did not reach significance in this study. Embryos were assessed at 48 hpf. n≥4 biological replicates using embryos from n≥2 breeding pairs. Vehicle was 0.08% phenol red+PBS in ultrapure water. Multiple comparisons were made between the vehicle and treatments using Fisher's exact test with Bonferroni's correction. * P<0.0085, *** P<0.00017, N.S.=not significant.

FIG. 26A and FIG. 26B present data for quantitation of immunofluorescence imaging of actin (FIG. 26A) and actinin (FIG. 26B) in adult heart segment A4. Statistical results of normalized cardiac fiber density (N=10). * P<0.05; ** P<0.01.

FIG. 27A, FIG. 27B, and FIG. 27C are optical coherence tomography (OCT) images for heart of control, ana3+/− and ana3−/− flies. Arrowheads indicate the end-diastolic diameter (EDD). Statistical analysis for EDD (μm) and percent fractional shortening (FS) obtained from the optimal computed tomography data is shown in FIG. 28A and FIG. 28B. Each datapoint represents the average of measurements from three heartbeats randomly selected within a two-second time frame for each fly. Center line=median; whiskers=1.5IQR for each genotype. * P<0.05.

FIG. 28A and FIG. 28B are graphs showing computed tomography data.

FIG. 29A is a pair of photomicrographs demonstrating that homozygous ana3 mutant showed minor microcephaly and severe early dissociated nerve cord. Bright field images of 3rd instar larvae brains from control (w1118) and ana3−/− flies. Scale bar: 100 μm.

FIG. 29B is a diagram of Drosophila melanogaster 3rd instar larvae brain.

FIG. 29C is a graph presenting data on quantitation of brain lobe volumes for control (w1118) and ana3−/− flies. Individual data points with group mean (horizontal bar)+SD are displayed for each genotype. Statistical significance (*) defined as P<0.05.

FIG. 30A is a heat map showing expression of genes related to CM maturation in control and iDCM-CMs.

FIG. 30B is a dot plot of fold enrichment (fe) of gene ontology (GO) pathways. up=upregulated in iDCMs; down=downregulated in iDCMs.

FIG. 30C is a plot showing the Shannon entropy score (relative maturation score) of control and iDCM-CMs.

FIG. 31A, FIG. 31B, FIG. 31C, FIG. 31D, FIG. 31E, and FIG. 31F are a set of photomicrographs showing that, in iPSCs, PCNT is colocalizes with γ-tubulin at the centrosome. Following cardiac differentiation, PCNT becomes partially redistributed to the perinuclear region of iPSC-CMs.

FIG. 31G, FIG. 31H, FIG. 31I, FIG. 31J, FIG. 31K, FIG. 31L, FIG. 31M, and FIG. 31N are a set of photomicrographs showing that the pericentriolar material components PCNT and PCM1 are redistributed to the perinuclear region in control iPSC-CMs, but not in RTTN mutant iPSC-CMs.

FIG. 31O is a graph showing a lack of any significant changes in primary cilia length.

FIG. 32 is a set of photomicrographs showing that rotatin (RTTN) is colocalized to the centrosomes in HEK293 cells, iPSCs, and iPSC-CMs. RTTN was detected using the SASY antibody, which is directed against the N-terminal region of RTTN. In HEK293 cells, SASY detected RTTN in both proliferating and quiescent cells; however, in iPSCs and iPSC-CMs, SASY detected RTTN predominantly in proliferating cells. The were no gross differences between RTTN localization in WT and cDCM iPSCs and iPSC-CMs, as detected by SASY.

FIG. 33A shows control CM and RTTN mutant CM as indicated.

FIG. 33B presents data for quantitation of PCNT distribution in D36 control and iDCM-CMs by a blinded observer with categories of centriolar, split, or perinuclear.

FIG. 34A shows representative control CM and iDCM CM as indicated. Scale bar=50 μm.

Quantitation of CMs was performed by a blinded observer and is presented in FIG. 34B.

FIG. 34C is a bar graph showing significant levels of concordance in control, iDCM, and GC-CMs.

FIG. 35 is a schematic detailing C19 treatment at differentiation day 30 (d30) control-CMs and iDCM-CMs.

FIG. 36A presents the quantitation of centriolar, split, and perinuclear MTOC localization in D32 CMs, performed by a blinded observer.

FIG. 36B shows representative images of untreated iDCM-CMs and iDCM-CMs treated with C19, as indicated. Scale bar=50 μm.

FIG. 36C is a set of boxplots showing % cell shortening in control, D40 iDCM, and iDCM C19 treated CMs, as indicated. Center line=median; whiskers=1.5IQR.

FIG. 37A is a drawing showing a working model of the CM maturation cascade. FIG. 37B and FIG. 37C are photomicrographs presenting data for microtubule regrowth assays (FIG. 37B=control; FIG. 37C=cDCM).

FIG. 38A and FIG. 38B are micrographs showing the defective microtubule network in RTRN mutant iPSC-CMs.

FIG. 38C and FIG. 38D are micrographs showing that control iPSC-CMs displayed a prominent network of thick microtubule (MT) fibers (a-tubulin, green) emanating from the perinuclear region. By contrast, cDCM iPSC-CMs displayed thinner, shorter, and fainter MT fibers without a clear organizing center. Insets show enlarged images.

### DETAILED DESCRIPTION OF EMBODIMENTS OF THE INVENTION

### 1. Overview

During cardiomyocyte (CM) maturation, the centrosome undergoes a dramatic structural reorganization where certain components, previously localized at the centrioles, become localized to the nuclear envelope. This developmental program has been associated with postnatal cell cycle exit but is otherwise relatively unstudied. Here, we describe an infant with congenital dilated cardiomyopathy (cDCM) whose impaired cardiac function and disrupted sarcomere and mitochondria structures were modeled using induced pluripotent stem cells (iPSCs). The causal gene encodes the centrosomal protein rotatin (RTTN), representing the first time a centrosome defect has been found to cause nonsyndromic dilated cardiomyopathy (DCM) in humans. Genetic knockdowns in zebrafish and Drosophila confirm an evolutionarily conserved requirement of RTTN for cardiac structure and function. Mutant iPSC-derived cardiomyocytes (cDCM-CMs) exhibited persistent localization of the pericentriolar material at the centriole, contrasting with programmed initiation of organization perinuclearly, and subsequent global microtubule network defects. In addition, a small molecule, C192, was identified that restored initiation of the perinuclear MTOC, and significantly improved the structure and function of the cDCM-CMs. In summary, this study demonstrated a novel role of RTTN in the cardiac centrosome required for structure and function, and suggests a potential therapeutic strategy for cDCM.

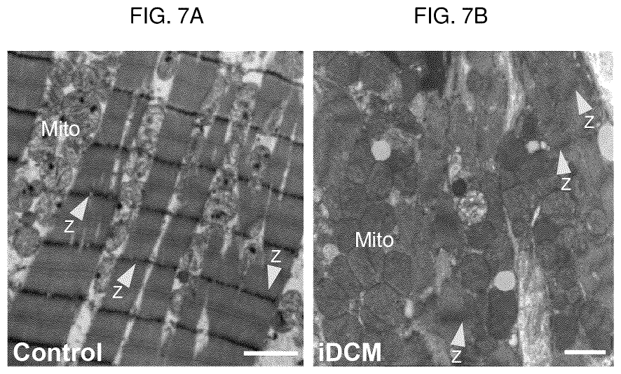

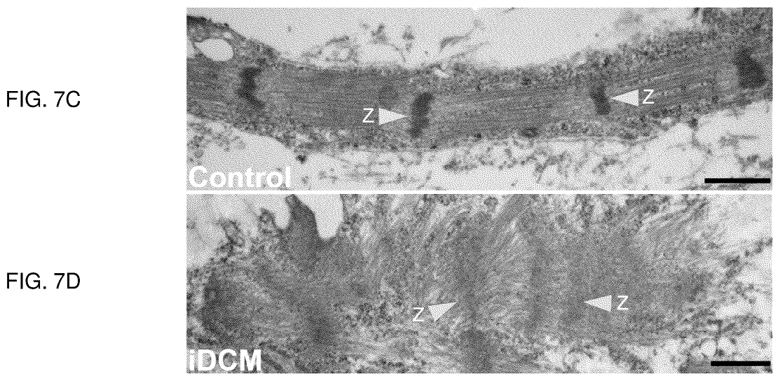

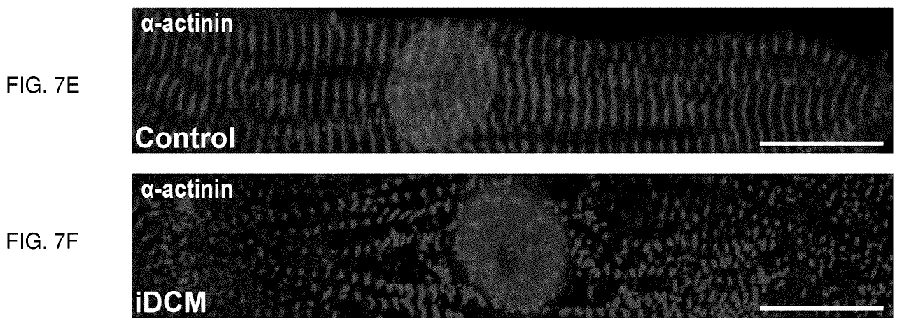

In this study, therefore, we incorporate iPSCs, CRISPR/Cas9, whole exome sequencing, single cell RNA sequencing, and multiple in vivo models to uncover biological insights from a case of infantile dilated cardiomyopathy. A male infant patient who presented with infantile dilated cardiomyopathy (iDCM) with a left ventricular ejection fraction of 18% is described. This patient underwent a successful heart transplant and does not presently exhibit any neurocognitive or neuromuscular deficits. The infant was born to non-consanguineous parents without a family history of cardiomyopathy. An endomyocardial biopsy and transmission electron microscopy (TEM) of the infant's heart revealed cardiomyocytes with shortage of myofilament structures, pleomorphic mitochondria, and indistinct Z-lines. See FIG. 7A. Given that multiple aspects of cardiac structure and function were significantly affected shortly after birth in this case of iDCM, whether this patient's condition had a genetic etiology was investigated.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “Hippo pathway activator compound” refers to one or more of a group of compounds that activate the Hippo signaling pathway, which regulates cell proliferation and apoptosis, for example by activating the protein kinase Hippo (Hpo). Examples of such compounds include, but are not limited to Verteporfin, Compound 13, CAY10583, CA3, SBI-115, and C19.

### 3. Summary of Results

Compound heterozygous mutations were identified in the centrosomal component rotatin (RTTN) as the causal gene for infantile dilated cardiomyopathy through dysregulating a process known as centrosome reduction in human cardiomyocytes. Such defects in centrosome reduction have implications of broad cardiomyocyte biology-sarcomere structure, mitochondrial dynamics, cell cycle- and are caused by disorganization of microtubule networks, highlighting a non-canonical role of the centrosome in cardiomyocyte biology.

Infantile dilated cardiomyopathy has multiple etiologies, so this study links a novel gene (RTTN) and a relatively unstudied developmental pathway (centrosome reduction) to human heart disease for the first time. A multi-pronged approach thus was used to uncover the biological etiology of infantile dilated cardiomyopathy and to identify therapeutic targets to overcome the defect. This paradigm is applicable across disciplines and fields to discover cures to human disease.

Whole-exome sequencing and CRISPR/Cas9 gene knockout/correction identified the centrosomal protein rotatin (RTTN) as the causal gene underlying the patient's condition, representing the first time a centrosome defect has been implicated in a non-syndromic dilated cardiomyopathy (DCM). Genetic knockdowns in zebrafish and Drosophila confirmed an evolutionarily conserved requirement of RTTN for cardiac structure and function. Single cell RNA-sequencing of iDCM-CMs showed impaired maturation of iDCM-CMs, which underlie the observed CM structural and functional deficits. Interestingly, we also observed persistent localization of the centrosome at the centriole, contrasting with expected programmed perinuclear reorganization, which led to subsequent global microtubule network defects. Finally, we identified a small molecule that restored centrosome reorganization, and improved the structure and contractility of iDCM-CMs.

This study demonstrates a case of human disease caused by a defect in centrosome reduction, found a novel role for RTTN in perinatal cardiac development, and identified a potential therapeutic strategy for centrosome-related iDCM.

### 4. Embodiments of the Invention

### A. Introduction

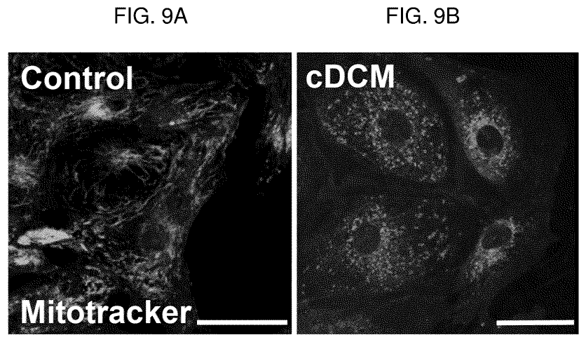

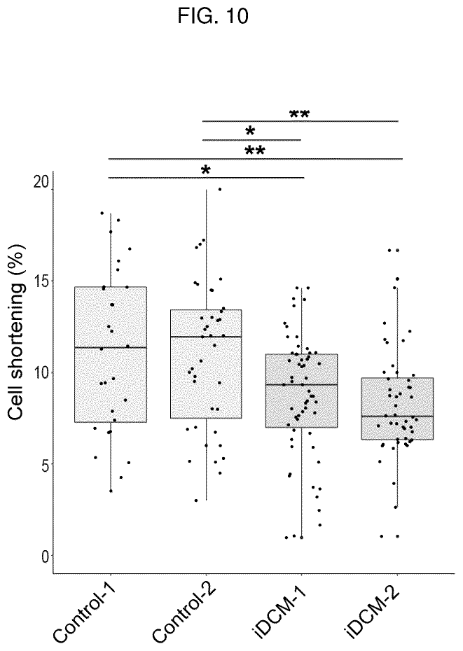

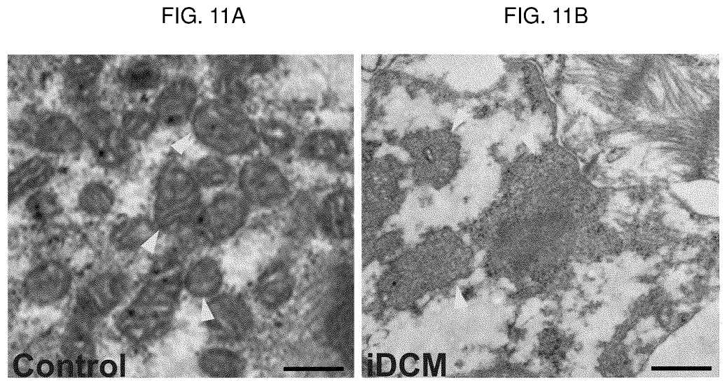

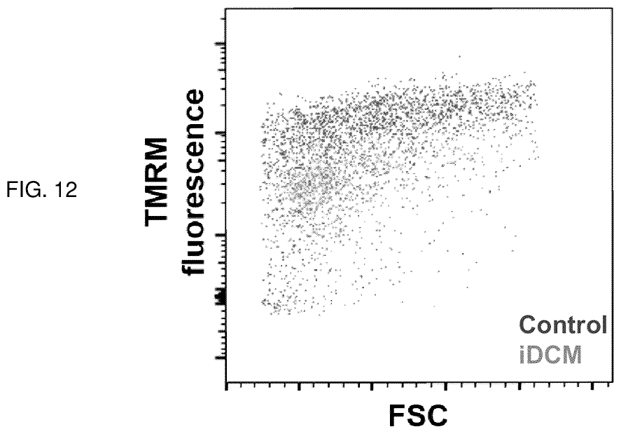

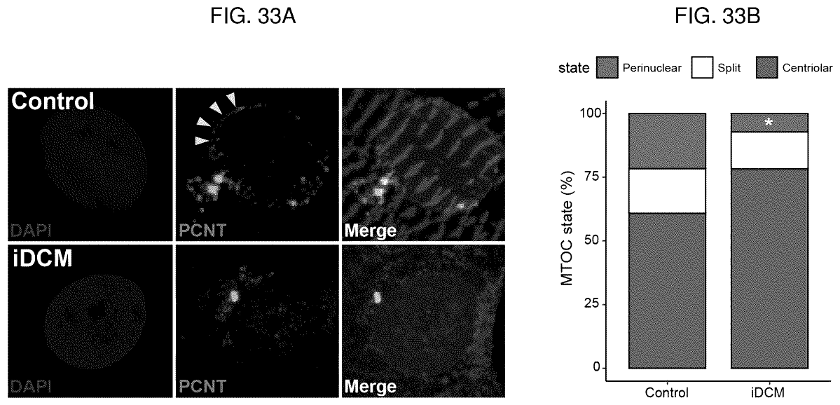

Centrosomes are dynamic organelles that undergo a type of programmed reorganization, termed as centrosome reduction, during cardiac differentiation. This study reports a defective centrosome component as the cause of nonsyndromic cardiomyopathy in humans (a syndromic cause of variable cardiomyopathy is observed in Alstrom syndrome). Here, it is shown that mutations in the centrosome component RTTN resulted in CM-specific defects in centrosomal reorganization during cardiac maturation. In the mutant CMs, the failure of the centrosome to reorganize and establish the nuclear envelope as the major microtubule organizing center resulted in a profoundly defective microtubule network (see, for example, FIG. 33B). Because the microtubule cytoskeleton is critical for the maintenance of cellular structures, including vital roles in mitochondrial motility, fission, and fusion, a defective microtubule network can be an important cause for both the mitochondrial and sarcomeric defects observed in RTTN mutant CMs, resulting in defective mitochondrial respiration and contractility (see FIG. 7, FIG. 9, FIG. 11, and FIG. 12. Further, control iPSC-CMs displayed a prominent network of thick microtubule (MT) fibers (a-tubulin, green) emanating from the perinuclear region. By contrast, cDCM iPSC-CMs displayed thinner, shorter, and fainter MT fibers without a clear organizing center. Insets show enlarged images.

Previously, the precise role of RTTN in the cardiac centrosome has been unknown, and it is unclear why the RTTN mutations described here only cause cardiac defects, in contrast to other published RTTN mutations, which cause CNS defects associated with centrosome amplification. There has been at least one known patient carrying a RTTN mutation with a restrictive cardiomyopathy in addition to a co-occurring CNS defect, however. Given the highly dynamic RTTN localization noted using SASY and the importance of alternative splicing in PCNT transition during cardiac differentiation, this study was performed based on the hypothesis that RTTN likewise can undergo substantial alternative splicing, resulting in tissue-specific isoforms with distinct roles in centrosome dynamics that can affect the development of specific organs.

Although much attention has been paid to the association of centrosome reduction with cell cycle arrest, this study demonstrates that tissue-specific and developmentally programmed centrosome reorganization is essential for proper CM structure and function. Because centrosome dynamics play fundamental roles in CM cell cycle exit, structure, and function, a better understanding of this tissue-specific, developmentally programmed process is highly relevant to cardiac regenerative therapy efforts and forms the basis of therapy for conditions involving cardiomyocyte defects or problems.

The male infant patient discussed herein was born full term to a G2P2 mother following an uncomplicated gestation. He had no syndromic features and was discharged home after an uncomplicated initial hospital stay. At 3 months of age, the patent presented to the referring hospital in Tennessee with failure to thrive. At that time, the patient was found to have a dilated left ventricle (left ventricular end-diastolic index of 63 ml/m²) and a markedly reduced ejection fraction of 18% (normal>55%) with global hypokinesis. He was ruled out for left ventricular noncompaction and was found to have normal coronary anatomy.

A global metabolic panel was negative for primary metabolic disorders, and the genetic consultation revealed no chromosomal abnormalities. There was no family history of congenital heart disease, sudden cardiac death, arrhythmias, neurologic disorders, metabolic disorders, or unexplained childhood deaths. The patient's maternal grandmother had an “enlarged heart.” His older sibling was a healthy teenager. Heart biopsy performed at 4 months of age was negative for acute inflammation, infiltrative processes, and viral inclusions. The biopsy was notable for severe loss of myofilaments and pleiotropic, dysmorphic mitochondria. He was critically ill for 4 months, eventually requiring extracorporeal membrane oxygenation before heart transplantation at 7 months of age. Following the transplantation, the patient has done well, without clinically significant neuromuscular or cardiac issues.

### B. Small Molecules

The Hippo pathway is a tightly regulated signaling pathway involved in controlling cell growth, proliferation, and apoptosis (regulating tissue size, maintaining organ homeostasis, and preventing cancer development). It is activated by stimuli such as cell-cell contact, mechanical stress, and extracellular signals. The core components of the Hippo pathway consist of a kinase cascade that blocks the activation of the transcriptional co-activators YAP (Yes-associated protein) and TAZ (transcriptional co-activator with PDZ-binding motif). When the Hippo pathway is off, the downstream effectors, YAP and TAZ, translocate to the nucleus and bind to transcription factors to regulate gene expression, but when the Hippo pathway is activated, YAP and TAZ are inactivated.

Here, we specifically describe the use of one or more of small molecule C19 and other Hippo pathway activators to restore cardiac structure, function, and metabolism in the setting of congenital dilated cardiomyopathy, and its further use as a therapeutic compound in congenital and adult cardiomyopathies. The small molecule C19, a Hippo pathway activator, induces centrosome reduction in a cardiomyocyte specific manner to induce maturation in the cardiomyopathy. While the data presented here relates to C19, any compound with that same function, i.e., activating the Hippo pathway to promote cell cycle exit and/or promote centrosome reduction will have the same effect and use. Thus, any Hippo pathway activator can promote cardiomyocyte maturation, permanently reversing heart diseases characterized by impaired cardiomyocyte maturation.

Examples of Hippo pathway activators include c19, Verteporfin, Compound 13, CAY10583, CA3, and SBI-115. Verteporfin was originally developed as a photosensitizer for photodynamic therapy of macular degeneration, but it was later found to activate the Hippo pathway by inhibiting the activity of the YAP/TAZ transcriptional co-activators. Compound 13 was identified in a screen for compounds that could activate the Hippo pathway by inhibiting the activity of the protein kinase LATS1/2, which is a key negative regulator of the pathway. CAY10583 also was identified in a screen for compounds that could activate the Hippo pathway by inhibiting the activity of LATS1/2. CA3 was found to activate the Hippo pathway by inhibiting the activity of the protein phosphatase PP1A, which is a negative regulator of the pathway. SBI-115 was identified in a screen for compounds that could activate the Hippo pathway by inhibiting the activity of the protein phosphatase PP2A, which also is a negative regulator of the Hippo pathway. Any of these compounds are contemplated for use with the invention in the treatment methods described. Further, derivatives, salts, and the like of C19 and these other compounds can be used with the invention.

It is contemplated that the Hippo pathway activator compounds can be administered as a monotherapy, or in combination with one or more activators of the Wnt signaling pathway. Examples of Wnt signaling pathway activators include the Wnt ligand, Prostaglandin E2, Norrin, and R-Spondin2.

### C. Conditions and Subjects to be Treated

This invention is contemplated for use as a therapeutic method for congenital, infantile, and adult cardiomyopathies such as dilated, hypertrophic, and left ventricular non-compaction cardiomyopathies. Examples of such conditions specifically include, but are not limited to: infantile or congenital dilated cardiomyopathy, acquired dilated cardiomyopathy, left ventricular non-compaction cardiomyopathies, other pediatric dilated cardiomyopathy, heart failure secondary to congenital heart disease, and the like.

Congenital and adult heart disease are leading causes of death among infants and adults, respectively, and many of these conditions arise due to failing cardiomyocytes. Administration of the inventive treatment methods facilitate restored cardiomyocyte structure and function in diseased hearts, improving contractility as a result. Transient treatment with C19 and other compounds with similar properties can be sufficient to reverse cardiomyopathy characterized by myocyte immaturity. In general, this invention can be used in any patient suffering from the conditions discussed above, but is most useful for treatment of pediatric patients in intensive care with heart failure or on life support (including ECMO or on the heart transplant list). Preferably, the patient has had a heart biopsy that rules out myocarditis.

Currently, there is no known therapeutic for congenital dilated cardiomyopathy. Consequently, affected patients require heart transplantation, for which there is an extremely limited supply of donor hearts. Thus, those suffering from congenital cardiomyopathies experience high levels of morbidity and mortality. The inventive methods provide a non-invasive, cardiomyocyte-specific, correction of the comprehensive cell biology of diseased cardiomyocytes, reducing the burden of transplant on patients and hospitals, and restoring cardiac function. The invention therefore is a more widely accessible form of treatment for the more than 200,000 people diagnosed with cardiomyopathy in the United States every year.

The conditions specifically contemplated for use with the invention include, but are not limited to, infantile or congenital dilated cardiomyopathy, acquired dilated cardiomyopathy, left ventricular non-compaction cardiomyopathies, other pediatric dilated cardiomyopathy, heart failure secondary to congenital heart disease, and the like.

### D. Methods

Once a child or infant is diagnosed with heart failure (specifically dilated cardiomyopathy but also due to other causes), the invention can be delivered orally, subcutaneously or intravenous for period of 1 to 14 days. The treatment can be initiated empirically, but can be aided by biopsy ruling out myocarditis and demonstrating defective centrosome reduction or other markers of cardiomyocyte immaturity, including disruption of microtubule network, sarcomere disarray (specifically the loss of Z-discs), dysmorphic mitochondria, and high cardiomyocyte proliferation index.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. General Information

Analysis was performed beginning with an infant patient who presented with a rare case of iDCM. Induced pluripotent stem cells (iPSCs) were derived from the patient to model iDCM in vitro. Whole exome sequencing was performed on the patient and his parents to search for causal gene analysis. CRISPR/Cas9 mediated gene knockout and correction in vitro were used to confirm whole exome sequencing results.

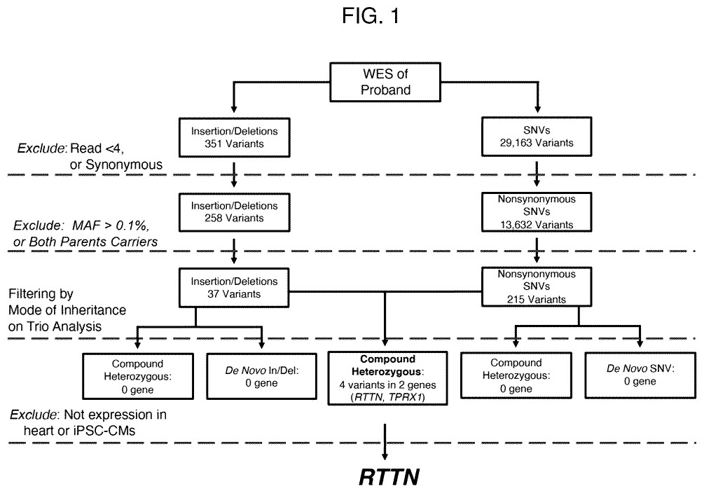

Whole exome sequencing (WES) of the proband and both parents was performed to identify a de novo mutation or compound heterozygosity. After filtering for a read depth>4 for quality and excluding synonymous variants, the proband's variant calling identified 258 indels and 13,632 nonsynonymous single-nucleotide variants (SNVs). Next, variants that were not rare (minor allele frequencies [MAF]>0.1%) and variants where both parents were heterozygous carriers or either parent was homozygous were excluded, resulting in 37 indels and 215 SNVs as candidates. Finally, a 3-fold filter of functional prediction (exclusion of benign prediction using polyphen2), expression in CMs, and validation by Sanger sequencing yielded a single gene candidate, RTTN (encoding rotatin). See FIG. 1.

Zebrafish and Drosophila models were utilized for in vivo validation of causal gene. Matrigel™ mattress technology and single cell RNA-sequencing were used to further characterize iDCM-CMs.

Human iPSC lines were generated from healthy control subjects using an episomal approach according to known methods validated previously. All protocols were approved by the Vanderbilt University Institutional Review Board and informed consent was given. scRNA sequencing data has been deposited in Gene Expression Omnibus (GSE184899) and full code can be found at: github.com/miyamoto5/cDCM.

### B. Patient-Derived Induced Pluripotent Stem Cell Derivation and Maintenance

Left ventricular tissues were taken from a 7-month-old patient with congenital dilated cardiomyopathy (cDCM), diced with a scalpel, and digested with collagenase IV (1 mg/ml, Invitrogen™) for 2 hours at 37° C. Primary fibroblasts were grown in fibroblast culture medium (Dulbecco's modified Eagle's medium (DMEM) with glutamine supplemented with 10% fetal bovine serum (FBS; Invitrogen™) in tissue culture dishes. The medium was changed every other day.

The episomal vectors pEP4EO2SCK2MEN2L and pEP4EO2SET2K were used to reprogram fibroblasts from the cDCM patient. They were cotransfected into ˜1.0×10⁶ patient-derived fibroblasts via nucleofection (VPD-1001 with program U-20, Amaxa™, Walkersville, MD). Transfected cells were directly plated onto 3×10 cm Matrigel-coated dishes (1:200) in fibroblast culture medium. After one day of transfection, the fibroblast medium was replaced with reprogramming medium supplemented with DMEM/F12, N-2 supplement (Invitrogen™), B-27 supplement (Invitrogen™), 100 ng/ml bFGF (Invitrogen™), nonessential amino acids (Invitrogen™), GlutaMAX (Invitrogen™), 0.1 mM β-mercaptoethanol, PD0325901 (Stemgent™), CHIR99021 (Stemgent™), A-83-01 (Stemgent™), hLIF (Millipore™), and HA-100 for 12 days. The medium was changed every other day. On day 13, the post-transfection medium was replaced with mTeSR1medium (Stem Cell Technologies™). Individual colonies with human embryonic stem cell-like morphologies were plated on 12-well plates (1 colony per well) at day 20-30.

The iPSCs were maintained in mTeSR1 medium (Stem Cell Technologies™) on 1:200 growth-factor-reduced (GFR) Matrigel (BD Science™) and passaged 1:10 or 1:12 every 3-4 days using 0.5 mM EDTA (Mediatech™). Cell lines were used between passages 13 and 75. All cultures (primary, pluripotent, and differentiation) were maintained with 2 ml medium per well in 6-well plates.

### C. Maintenance of iPSCs and Cardiac Differentiation

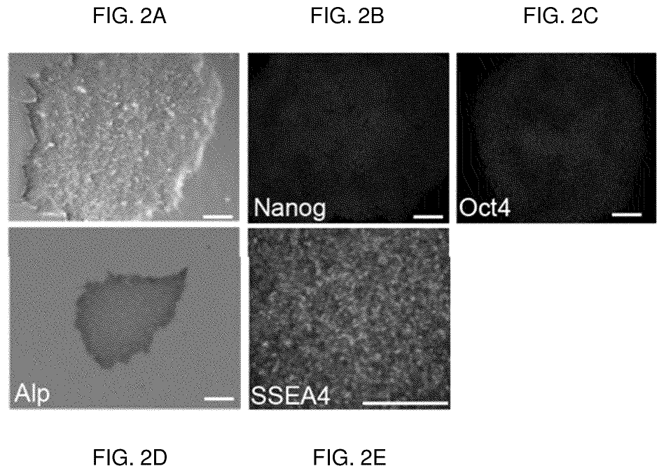

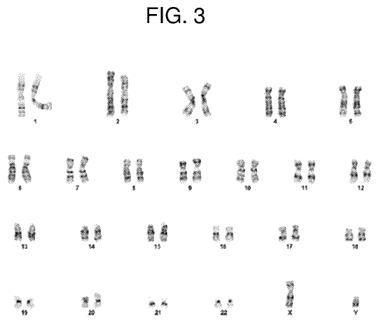

All iPSCs used in this study were positive for NANOG, Oct4, SSEA4, and alkaline phosphatase (see FIG. 2A through 2D) and were maintained on plates coated with GFR Matrigel (Corning™) in mTeSR1 (Stem Cell Technologies™). iPSCs were maintained at 37° C. with 5% carbon dioxide and 18% oxygen. Karyotype analysis of control iPSCs confirmed that there were no chromosomal abnormalities (see FIG. 3). Cardiac differentiation of control and cDCM iPSCs was performed using the known small molecule-based method. Control and cDCM iPSCs were grown for 4 days until they achieved 80˜90% confluence. Day 0 was designated at this point; the medium was changed to basal medium RPMI 1640 (Life Technologies™) plus B27 without insulin supplement (A1895601, Life Technologies™), supplemented with 6-8 μmol/L CHIR99021 (Selleck Chemicals™) for 2 days. On day 2, the medium was changed to RPMI 1640 plus B27 without insulin without CHIR99021. On day 3, the medium was changed to basal medium RPMI 1640 plus B27 minus insulin, supplemented with 5 μmol/L IWR-1 (Sigma™). Medium was changed on day 5 and every other day thereafter until day 11 to RPMI 1640 plus B27 without insulin.

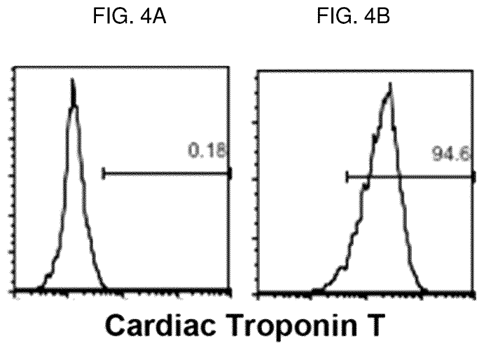

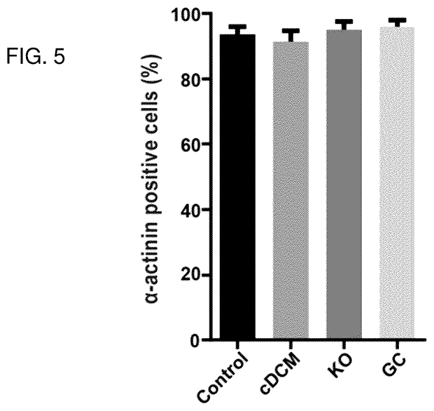

At about day 11-13, the medium was changed to metabolic selection medium consisting of basal RPMI 1640 without glucose (Life Technologies™) plus B27 without insulin. On day 14, the medium was changed to RPMI 1640 medium with B-27 supplement (Invitrogen™), and 1% Pen-Strep (Life Technologies™) until experiments were performed. More than 90% of the resulting cells were positive for cardiac troponin T (see FIG. 4A and FIG. 4B) or α-actinin (see FIG. 5), and were cultured in in RPMI 1640 with 2% B27 complete (Invitrogen) with 1% penicillin/streptomycin (Gibco™) for 30 days.

### D. Cell Shortening Analysis

Matrigel mattress substrates were generated as described. Briefly, 1 μL of undiluted Matrigel solution was arrayed on a Mattek dish (Fisher Scientific™) and incubated for 10 minutes. iPSC-CMs dissociated by TrypLE Express (Invitrogen™) were seeded on solid undiluted Matrigel. Video edge detection was used to assess the contractility of contracting CMs by measurement of relaxed and contracted length. CMs were visualized by a Hamamatsu-ORCA camera with 16 to 17 fps using a 20× objective. Individual cells in RPMI1640 containing 2% complete B27 supplement (Life Technology™) in the presence of 1% penicillin/streptomycin were recorded. The recorded files were converted into AVI files and analyzed using ImageJ (Multi Kymograph™). The numbers of iPSC-CMs used in this study: 28 and 39 cells for control-1 and -2; 59 and 48 cells for cDCM-1 and -2; 43 and 29 cells for KO-1 and -2; and 32, 35, and 42 cells for GC-1, -2, -3. Cells that did not shorten were excluded from the analysis.

### E. Flow Cytometry Analysis

iPSC-CMs maintained for 30 days were detached with TrypLExpress (Invitrogen™). They were singularized by slowly pipetting up and down and filtered using 2 μm cell strainers (Thermo Fisher™). 5×10⁵-1×10⁶. Cells were fixed in 1% paraformaldehyde at room temperature for 10 minutes, permeabilized by FACS buffer (PBS without calcium or magnesium, 1% FBS, 0.1% sodium azide) with 0.1% saponin. After washing, the cells were blocked with FACS buffer for 30 minutes and then treated with Troponin T antibodies (Santa Cruz Biotechnology™) diluted at 50 μl/sample in FACS buffer plus 0.1% Triton X-100. Cells were incubated with primary antibodies for 1 hour at 4° C., washed once in 3 ml FACS buffer plus 0.1% Triton X-100, and then centrifuged. Secondary antibodies specific to the primary antibody IgG isotype were diluted in FACS buffer plus 0.1% Triton X-100 for a final sample volume of 100 μl at 1:1,000 dilution. Cells were incubated for 30 minutes in the dark at 4° C.; washed in FACS buffer plus 0.1% Triton X-100; and resuspended in 300 μl FACS buffer for analysis. The resulting data were analyzed using FlowJo v8.5.2 and reported data are based on 10,000 gated events.

### F. Mitochondrial Morphology and Function

To observe the mitochondrial morphology and function of iPSC-CMs, iPSC-CMs were treated with either 50 nM Mitotracker Green FM (Molecular Probes™) or/and 2 nM tetramethylrhodamine methyl ester perchlorate (TMRM) [5] (Sigma™) in RPMI 1640 without phenol red with 2% B27 complete (Invitrogen™) with 1% penicillin/streptomycin (Gibco™) for 30 minutes. After washing, the cells were incubated for one hour in nonphenolic media without Mitotracker and TMRM to remove unbound TMRM and Mitotracker. Some cells were used for TMRM imaging and others were used for quantitative analysis by flow cytometry. TMRM signals were limited by low green and high red fluorescence due to autofluorescence.

### G. CRISPR/Cas9 Expression Vector Construction

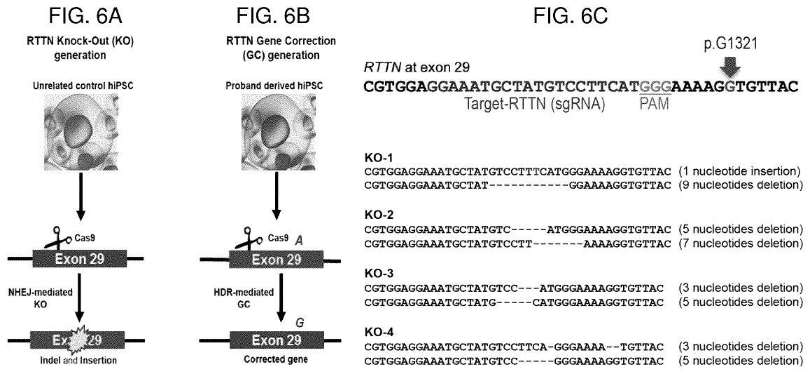

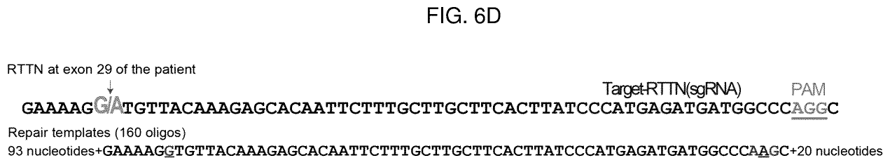

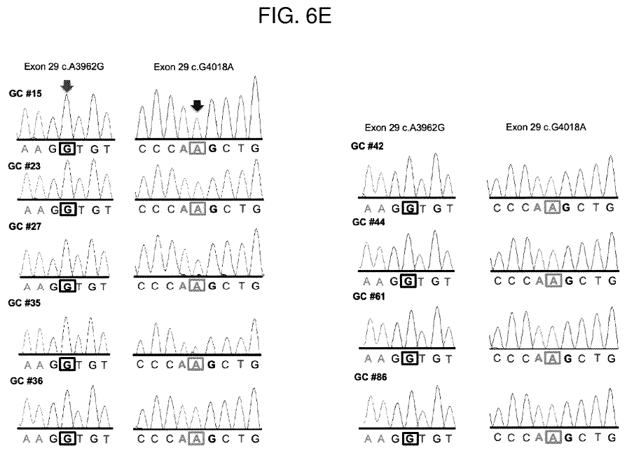

Single-guide RNAs (sgRNAs) were designed using the Zhang lab CRISPR design tool. sgRNA sequence (see FIG. 6B) were cloned into pSpCas9 (BB)-2A-puro (PX459) (Addgene™) plasmid, as described. Insertion of the sgRNA was confirmed by Sanger sequencing.

### H. Genome Modification in Control and Patient-Derived iPSCs

iPSCs were singularized using 0.5 mM EDTA in DPBS without calcium chloride and magnesium chloride and passed through a 30-μm filter. We delivered 10 μg of the Px459 plasmid containing the sgRNA insert to 2×10⁶ control iPSCs by electroporation (Epi5 Episomal iPSC Reprogramming Kit, ThermoFisher™). For genome correction, cDCM iPSCs were transfected with the sgRNA in the Px459 plasmid and 10 μmol of the 160-bp single-stranded oligomers. To expand individual clones post-delivery, iPSCs were grown on 10-cm plates coated with growth factor-reduced Matrigel (1:200 dilution) and treated with puromycin (1 μg/ml) for screening 36 hours after transfection. Puromycin-resistant clonal colonies were picked and manually transferred to Matrigel-coated 24-well plates for expansion. Each colony was passaged using 0.5 mM EDTA for line maintenance and DNA extraction. DNA was isolated using QuickExtract solution (EpiCentre™) for PCR amplification of exon 29 of RTTN. Screening of genome-corrected colonies was done by digestion with ScrfI (New England Biolabs), which selectively cut the cDCM sequence but not the genome-corrected sequence due to altered nucleotides (G4018A) in the repaired sequence. Gene editing was confirmed in all positive clones by Sanger sequencing. KO and GC iPSCs were maintained with mTeSR1 (Stem Cell Technologies™) media.

### I. Cas9 Off-Target Analysis

Predicted off-target tool were amplified by PCR and evaluated by Sanger sequencing. The genomic location, nearest gene, and primers used for amplification are presented in Table 2 and Table 3, below.

TABLE 2
Predicted CRISPR-Cas9 off-target sites
against the sgRNA of KO lines.

| Potential off- | Number of | | | |
|---|---|---|---|---|
| target sequence, | mismatches | | | SEQ |
| forward primer, and | (variable | | Gene | ID |
| reverse primer | nucleotide) | Locus | name | NOs |
| AGCAATGCTGTTTCCTTCATAAG | 4MMs [1:3:10:12] | chr15: | SNRPB2 | 14 |
| GAGACCCAGGCAGGATCTAA | | 63918371 | | 15 |
| GCAAGCTTAGCACTGGCAAC | | | | 16 |
| GGCAATGTTCTGTCCTTCAGCAG | 4MMs [3:8:10:20] | chr1: | PTPRF | 17 |
| TGAGGGATTTTAGCCTCTTCC | | 44088160 | | 18 |
| ACGACCAGGGATGAGTGTTC | | | | 19 |
| CAAAAGGCAATGTCCTTCATCAG | 4MMs [1:2:6:9] | chr19: | ZAF528 | 20 |
| GTGCAACGGCTTGTAAATGA | | 52920543 | | 21 |
| TTTCATGGTCAGTTGGTGGA | | | | 22 |
| GGAAGCCCTATATCCTTCATTGG | 4MMs [5:6:7:12] | chr15: | AAGAB | 23 |
| GGTGGAACATGTTTTCCTTAAA | | 67493281 | | 24 |
| CCTTCATCCCATATGTCCCTA | | | | 25 |

TABLE 3
Predicted CRISPR-Cas9 off-target sites against the sgRNA of GC lines

| Potential off- | Number of | | | |
|---|---|---|---|---|
| target sequence, | mismatches | | | SEQ |
| forward primer, and | (variable | | | ID |
| reverse primer | nucleotide) | Locus | Gene name | NOs |
| ATCCCAAGGGAAGATGGCCCCAG | 3MMs [7:9:12] | Chr11: | CD248 | 26 |
| CAGGTCCCCTGTGTCTCCT | | 66082539 | | 27 |
| ACGTTGGCACCAGGAGTG | | | | 28 |
| CTCCCAGGAAATGATGGCCAGAG | 4MMs [1:7:10:20] | ChrX: | OPL1MW | 29 |
| GGAAATGCCCAGTGTCTGTT | | 153418426 | | 30 |
| AGTCTGACCCTGCCCACTC | | | | 31 |
| CTCCCAGGAAATGATGGCCAGAG | 4MMs [1:7:10:20] | ChrX: | PCSK6 | 32 |
| GCTTGTTGGCTCCAGTCTTC | | 153455556 | | 33 |
| GCCCCAGTGGTCATACATTT | | | | |
| | | | | 34 |
| CTCCCAGGAAATGATGGCCAGAG | 4MMs [1:7:10:20] | ChrX: | LINC00339 | 35 |
| TGGTCTGTTTTTGCTGATTCTT | | 153492674 | | 36 |
| CCGGGAGGCTTTCTAGCAT | | | | 37 |
| GTCACATGTGAGGATGGCCCAGG | 4MMs [1:4:9:12] | Chr15: + | PPFIA3 | 38 |
| CCCCGACATGACTCTTTGAA | | 101845019 | | 39 |
| TCTCCTCCCAAGTCCTTGTC | | | | 40 |

### J. Transmission Electron Microscopy Analysis

Specimens were processed for transmission electron microscopy (TEM) and imaged in the Vanderbilt University Cell Imaging Shared Resource Electron Microscopy facility. For embedding, cells on coverslips were fixed in 2.5% glutaraldehyde in 0.1 M sodium cacodylate buffer, pH 7.4 at room temperature (RT) for 1 hour and then transferred to 4° C. overnight. Samples were washed in 0.1 M cacodylate buffer, incubated for 1 hour in 1% osmium tetroxide at room temperature, and then washed with 0.1 M cacodylate buffer. Coverslips were rinsed with water and then contrasted with 1% potassium ferrocyanide for 15 minutes at room temperature. Coverslips were then washed with water and subsequently dehydrated through a graded ethanol series, followed by three exchanges of 100% ethanol. Next, samples were incubated for 5 minutes in 100% ethanol and then in propylene oxide (PO), followed by 2 exchanges of pure PO. The samples were then infiltrated with 25% Epon 812 resin and 75% PO for 30 minutes at RT. Samples were then infiltrated with Epon 812 resin and PO (1:1) for 1 hour at room temperature and then overnight at room temperature. The following day, the samples went through a 3:1 (resin: PO) exchange for 3 to 4 hours and were then incubated with pure epoxy resin overnight. Samples were incubated in 2 more changes of pure epoxy resin and then allowed to polymerize at 60° C. for 48 hours.

For sectioning and imaging, 70- to 80-nm ultrathin sections were cut and collected on 250-mesh copper grids and post-stained with 2% uranyl acetate and then with Reynold's lead citrate. Samples were imaged using a Philips/FEI Tecnai T12 electron microscope at various magnifications.

### K. Immunostaining

For immunofluorescence imaging, iPSC-CMs were fixed, permeabilized, and blocked according to known methods. Samples were fixed in 4% paraformaldehyde for 10 minutes, permeabilized with 0.2% Triton X-100 in PBS without calcium and magnesium for 10 minutes, and then blocked with Protein Block (Biogenex™, KH112-9k) for 15 minutes at room temperature. The iPSC-CMs were incubated overnight at 4° C. in primary antibodies in Protein Block solution containing 0.2% Triton X-100. After multiple washes with 0.2% Triton X-100 in PBS, secondary antibodies specific to the primary antibody IgG isotype were diluted (1:1,000) in Protein Block solution containing 0.2% Triton X-100 and incubated at room temperature for 1 hour.

SASY staining was performed using the methanol method according to known methods. Briefly, human embryonic kidney (HEK) cells, iPSCs, and iPSC-CMs were incubated in cold 100% methanol at −20° C. for 10 minutes. After washing, the cells were blocked with Protein Block (Biogenex™, KH112-9k) for 90 minutes at 4° C. The cells were treated overnight at 4° C. in primary antibodies in Protein Block plus 0.5% Triton X-100. After thorough washing, the cells were treated with the secondary antibody corresponding to the species of the primary antibody for one hour at room temperature. After thorough washing, all immunostained cells were mounted in Vectashield™ (Vector Laboratories™) including DAPI, visualized by a Zeiss laser-scanning LSM 710 META Inverted confocal microscope or a Nikon A1 point laser confocal microscope and analyzed with NIS-Elements BR3.0 and Fiji software.

For Drosophila staining, female adults (day 1) were collected and their hearts were processed as follows, in brief: the heart was dissected and fixed in 4% paraformaldehyde (PFA) (Polysciences™) in 0.1 M phosphate buffer (pH 7.4) for 20 minutes. The fixed specimen was permeabilized in 0.1% Triton X-100 in PBS (PBST) for 10 minutes; then blocked in PBST with 2% BSA for 1 hour and incubated overnight with primary antibodies at 4° C. For staining of the adult heart, phalloidin conjugated Alexa Fluor 568 (Thermo Fisher Scientific™) was diluted at 1:1,000 and incubated with dissected heart overnight at 4° C. Cy3-conjugated secondary antibodies (1:1,000; Thermo Fisher Scientific™) were used to recognize the actinin primary antibodies. All the fly hearts were imaged by a 63x Plan-Apochromat 1.4 N.A. oil objective (LSM900; ZEISS). For quantitation and comparison of cardiac fiber intensity, same parameters were used across all conditions, set to avoid oversaturation. Each image was adjusted to get the heart tube at the middle horizon position, transferred to 8-bit image, and mean gray value of each heart tube was measured.

### L. Antibodies

Antibodies used in this study were: cardiac Troponin T (Invitrogen, MA5-12960 and Abcam™, ab45932); sarcomeric α-actinin (Sigma™, A7732); α-tubulin (Invitrogen™, 62204); PCNT (Abcam™, mAbcam28144 and ab4448); PCM1 (Santa Cruz™, sc-398365); γ-tubulin (Sigma™, T-6557); rotatin (Santa Cruz I-20, sc-85129 and N-14, sc-85130; ThermoFisher™, PA5-98136; R&D System™, MAB9966; and Abcam™, ab 122710 and ab113541); ac-tubulin conjugated with Alex Fluor 488 (Santa Cruz™) and anti-Actinin (Developmental Studies Hybridoma Bank).

### M. Source of Blood from Proband and their Parents and DNA Isolation

Blood samples were obtained from the Core Lab for Translational and Clinical Research at Vanderbilt University Medical Center. The study was approved by the Vanderbilt Institutional Review Board, and written informed consent was obtained from the patient's parents. Blood samples were collected in BD Vacutainer EDTA tubes (Becton, Dickinson™ and Company, NJ) prior to heart transplantation. Samples were stored at −80° C. until used for DNA extraction. DNA was extracted from these specimens using Qiagen™ DNeasy Blood & Tissue Kit (Qiagen™) according to the manufacturer's instructions.

### N. Whole-Exome Sequencing of Trios (Patient and Both Parents)

We performed Trio Whole-Exome Sequencing (WES) of the proband and his parents at the Vanderbilt Advanced Genomics Core Facility using an Illumina Hi Seq 2500 with 100× coverage. Data were processed through Illumina's CASAVA v1.8.2 pipeline. We conducted thorough quality control based on the multistage quality control protocol developed at Vanderbilt. No sequencing quality concerns were observed. Alignments were performed using Burrows-Wheeler Alignment against human genome reference UCSC HG19. We marked duplicates using Picard™, and then performed local realignment and recalibration using the Genome Analysis Toolkit (GATK). Somatic mutations were inferred using the GATK analysis pipeline. The results were further filtered based on GATK's best practices. Annotations of single-nucleotide variants were performed using ANNOVAR.

### O. Confirmatory Sanger Sequencing of Variants Identified from WES

ANNOVAR variant calling identified 351 insertion/deletions and 29,163 single-nucleotide polymorphisms (SNPs) in the Trio analysis of the proband and his parents. Given the lack of family history of heart disease and nonconsanguinity, multiple filtering schemes were applied for hypotheses including de novo mutations, sex linkage, and compound heterozygosity. Based on filters of functional predictions (Polyphen-2), allele frequency (MAF<0.1%), and zygosity of SNPs in the parents, and the mode of inheritance, we arrived at four de novo, two X-linked, and two compound heterozygous mutations (see Table 4, below).

TABLE 4
Incorporating the iPSC-CM expression profile (RNAseq) and PolyPhen2 pathogenicity
prediction algorithm further narrowed candidate causal genes to RTTN.

| | Candidate | Amino Acid | | PolyPhen2 | Express (in |
|---|---|---|---|---|---|
| Type | Gene | Change | Aliases | Score | iPSC-CMs) |
| De novo | FRGC | S62X | FSHD Region Gene 2 | n/a, stop gain | No |
| | | | Family | | |
| De novo | ZNF718 | I11V | Zinc Finger Protein 718 | Benign | Yes |
| De novo | RPS6KA4 | L769P | Ribosomal Protein S6 | Possibly | Yes |
| | | | Kinase | deleterious | |
| De novo | RPS6KA4 | S772P | Same | Benign | Yes |
| De novo | BAGE3 | I63T | B Melanoma Antigen | Benign | No |
| | | | Family member 3 | | |
| X-linked | H2BFWT | I170fs | H2B Histone Family | n/a, frameshift | No |
| | | | | deletion | |
| X-linked | EFHC2 | G593E | EF-Hand Domain (C- | Benign to | Yes |
| | | | terminal) containing 2 | possibly | |
| | | | | damaging | |
| X-linked | KCND1 | T512P | Shal-type potassium | Benign | Yes |
| | | | Voltage-gated Channel | | |
| X-linked | MPP1 | M163T | Membrane Protein | Benign | Yes |
| Compound Het: | RTTN | 1921_1925 del | Rotatin, conserved | n/a, conserved | Yes |
| Del & SNP | RTTN | G1320D | KELSI deleted | 5AAs deleted | Yes |
| | | | Rotatin | possibly | |
| | | | | damaging | |
| Compound Het: | TPRX1 | 215_219 del | Tetra-peptide Repeat | | No |
| Del & SNP | TPRX1 | 206_210 del | Homeobox Protein | | No |
| | | | Tetra-peptide Repeat | | |
| | | | Homeobox Protein | | |

Finally, we validated variants by Sanger sequencing and assessed expression of the transcripts in heart tissue and iPSC-CMs. Based on these criteria, we arrived at a single putative causal gene, RTTN.

### P. Zebrafish Injections

Single mating pairs were bred overnight, and embryos were kept at 28.5° C., consistent with standard practices. Injections were performed using an Eppendorf™ FemtoJet 4i microinjector. Needles were pulled using a Sutter Instrument Co. Flaming/Brown Micropipette Puller Model P-97 with filaments from World Precision Instruments (Item #TW100F-4) (heat=90, pull=30, and velocity=7). The translational blocking morpholino oligonucleotide 5′-TCTTTATGAGTGACGATAACTCCAT-3′ (SEQ ID NO: 41) was purchased from GeneTools™. mRNA was generated from pcDNA-dCas9 (Addgene™) using the mMESSAGE mMACHINE T7 ULTRA Transcription Kit from Invitrogen™. Alt-R CRISPR-Cas9 sgRNA, sgRNA #1: 5′-ATATTTCATTATCCTACAAG-3 (SEQ ID NO:42) and sgRNA #2: 5′-TTTTCCGGAGGTTCCAATGC-3′ (SEQ ID NO:43) were purchased from Integrated DNA Technologies. Wild-type NHGRI embryos were injected at the 1-2 cell stage with MO (6 ng) or dCas9 mRNA (1 ng) with or without the CRISPRi sgRNA (0.5 ng). At 8 hours post fertilization (hpf), E3 water was replaced with 0.003% 1-phenyl-2-thiourea (PTU) in E3, and the embryos remained in PTU until they were phenotyped for cardiac edema, and heart morphology at 48 hpf.

### Q. Drosophila Stocks and Breeding

The two mutant fly lines for ana3 were a gift from Dr. Jordan W. Raff (University of Oxford, UK). Each mutant line was crossed with w1118, Hand-GFP; Sp/CyO-actGFP to introduce the cardiac-specific marker (Hand) and GFP balancer as described previously. Flies were raised and maintained in a controlled environment at 25° C., unless otherwise noted, and fed with standard fly food.

### R. Determination of Heart Morphology and Function by Optical Coherence Tomography (OCT)

Three-day-old female flies were collected and anesthetized by CO₂. Flies were immobilized on coverslips using GelWax (Yaley Enterprises™) under a dissection microscope. Flies were allowed to recover for 10 minutes and were then examined by OCT (Model: TEL220C1, Thorlabs™) at room temperature. Two-dimensional B-mode images were obtained in the longitudinal direction to identify the cardiac chamber in the A4-5 abdominal segments and then the OCT image orientation was turned 90° to obtain the transverse images. M-mode OCT images were obtained in the transverse plane by stopping the scanning mirror at the midline and collecting continuous image data. After obtaining M-mode images, repeat B-mode transverse images were obtained to ensure proper anatomic localization throughout image acquisition. All images were calibrated with a 150-μm standard.

### S. Survival Curve Assay for Drosophila

Flies were maintained in a humidified, temperature-controlled environmental chamber at 25° C. and live flies were counted every day for the survival assay. Each survivorship curve represents data from over 50 flies, with three independent experiments performed, for 150 flies in total.

### T. Imaging of the Larval Fly Brain by Stereo Microscope

At the late 3rd instar stage, the larvae were collected and dissected at room temperature in 1λPBS. The brain lobe and nerve cord were rinsed with 1λPBS, then put on a slide for visualization and image capture using a Stereo microscope (ZEISS SteREO Discovery v20; ZEISS). To examine brain morphology, differential interference contrast (DIC) images were collected at 175× magnification. Brain volume was calculated using the radius obtained from the DIC images.

### U. scRNA-Sequencing

Control and DCM CMs were dissociated at differentiation day 35. Following culture and dissociation, we prepared samples for cell capture using the 10x chromium 3′ v3 workflow, and sequenced on a NovaseqS4 flow cell. We integrated between conditions using the SCTransform workflow implemented in Seurat. As active cell cycling has an impact on contractile gene expression, we filtered out actively cycling cells in addition to non-CMs for further analysis. After filtering, we began analysis on 4,671 control and 1,080 DCM CMs. We performed differential gene expression testing using the Wilcoxon Rank Sum test with Bonferroni-correction-adjusted p-values<0.05. Gene Ontology analysis was done using the Gene Ontology Resource online tool. Full code and data for single cell RNA sequencing analysis will be available upon publication.

### V. C19 Treatment

D25 control and cDCM-CMs were dissociated and plated on Matrigel mattress coated Mattek plates. At D30, 10 μM C19 for 48 hours. Sarcomere and cell size analysis were conducted on Matrigel mattress, 10 days after start of treatment with confocal imaging and ImageJ™ analysis. Contractility analysis was conducted using edge detection as previously described.

### W. Statistical Analysis

Statistical differences among more than 2 groups were assessed using one-way analysis of variance followed by Tukey correction. For zebrafish data, multiple comparisons were made between the vehicle and control or treatments using Fisher's exact test with Bonferroni's correction. For Drosophila, statistical analysis was performed by Prism 7 (GraphPad™ Software). Statistical data are expressed as mean±SD and Student t test was applied to determine the significance of any differences between the two groups. A value of P<0.05 was considered statistically significant. Some statistical differences among two groups were tested with two-tailed Fisher's exact test.

### Example 2: An iPSC Model Recapitulates the iDCM Phenotype

A male infant presented with infantile dilated cardiomyopathy (iDCM) with a left ventricular ejection fraction of 18%. He underwent a successful heart transplant and does not presently exhibit any neurocognitive or neuromuscular deficits. To test the hypothesis that this case of infantile dilated cardiomyopathy (iDCM) had a genetic etiology, induced pluripotent stem cells (iPSCs) were derived from the infant patient to generate iPSC-derived cardiomyocytes (CMs).

Two independent iPSC lines were generated from the patient and were compared to two independent iPSCs lines from healthy donors. Compared with CMs from healthy donors, the iDCM-CMs exhibited profound sarcomere defects, recapitulating the cardiac defects seen in the explanted heart. See FIG. 7A and FIG. 7B, which present TEM images of hearts. FIG. 7A is an electron micrograph of normal heart showing well-organized myofilaments, distinct Z-lines (Z; arrowheads) and mitochondria (Mito) with distinct cristae. FIG. 7B is an electron micrograph of the iDCM patient's heart showing severely disorganized myofibrils with indistinct Z-lines and dysmorphic mitochondria (Mito) without appreciable cristae. Scale bar=1 μm. See also FIG. 7C and FIG. 7D, showing the Z-lines in control and iDCM heart tissue. The iPSC-CM from a healthy control exhibited organized myofilaments with distinct Z-lines (Z; arrowheads) (FIG. 7C), whereas the iPSC-CM derived from the iDCM patient (FIG. 7D) exhibit disorganized myofibrils without distinct Z-lines (Z). Scale bar=0.5 μm.

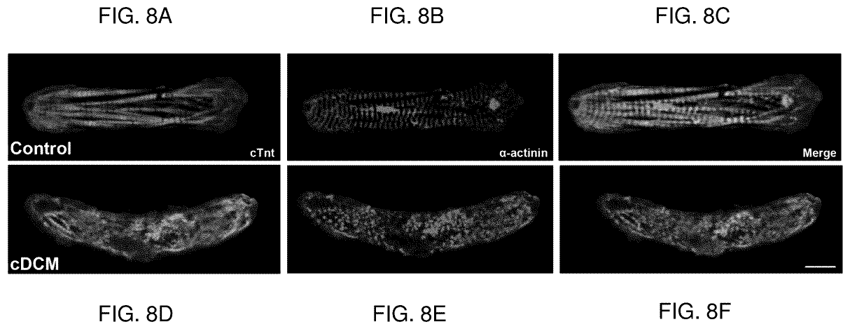

In addition, immunofluorescence imaging demonstrated that iDCM-CMs had disorganized Z-lines when compared with control-CMs. See FIG. 7E and FIG. 7F, which are representative immunofluorescence images of α-actinin staining of control and iDCM CMs, respectively. Scale bar=10 μm. See also FIG. 8A through FIG. 8F, and FIG. 9A and FIG. 9B, which show the sarcomere structure and mitochondrial function of control and cDCM iPSC-CMs. The cell area of iDCM-CMs was significantly decreased compared to Control-CMs.

Compared with control-CMs derived from 2 independent healthy control iPSC lines (Control-1, -2), iDCM-CMs from 2 independent iPSC lines (iDCM-1, -2) exhibited significantly reduced cell shortening (˜11% vs˜8%). p value by one-way ANOVA and post-hoc Tukey *p<0.01, ** p<0.05 (n=28). Control 1-CMs, 39 Control 2-CMs, 59 iDCM 1-CMs, 48 iDCM 2-CMs. Center line=median; whiskers=1.5IQR.

To observe mitochondrial morphology of iDCM-CMs, transmission electron microscopy (TEM) was used. Grossly dysmorphic mitochondria were observed, as shown in FIG. 11A and FIG. 11B. The TEM images of iPSC-CMs are focused on mitochondria. FIG. 11A shows easily distinguishable mitochondria of control iPSC-CMs with distinct cristae, whereas mitochondria from iDCM-CMs (FIG. 11B, arrowheads) appear larger and swollen, without clear cristae. Scale bar=0.5 μm.

Then, control-CMs and iDCM-CMs were stained with the vital mitochondrial dye MitoTracker™. MitoTracker™ staining of healthy control-CMs revealed interconnected networks of elongated mitochondria. Consistent with the TEM findings, mitochondria in the iDCM-CMs were grossly abnormal, often appearing globular and punctate. Staining with tetramethylrhodamine (TMRM), a vital dye that measures the mitochondrial membrane potential, indicated that iDCM-CMs had decreased mitochondrial membrane potential compared with control-CMs. See FIG. 12, which presents fluorescence-activated cell sorting analysis, demonstrating that iDCM-CMs have quantitatively lower TMRM uptake and hence lower mitochondrial membrane potentials than control iPSC-CMs. The TEM images of CMs were acquired at 45 days of differentiation. Other studies were done at 35 days of differentiation.

Additional studies showed that proliferation of iDCM-CMs was increased when compared to control-CMs at differentiation day 21 and 36. This recapitulation of the patient's phenotypic and functional deficits by patient-derived CMs indicates a genetic root cause for the patient's iDCM.

### Example 3: Mutations in RTTN Cause iDCM

To identify the causal mutation underlying this case of iDCM, whole-exome sequencing analysis of the patient and his parents was carried out. This analysis did not reveal pathogenic or likely pathogenic variants in any of the 123 genes known to be associated with cardiomyopathy (see Table 1, above).

Given the lack of a family history of cardiomyopathy and the nonconsanguinity, the causal mutation(s) most likely were either a de novo autosomal dominant mutation or rare compound heterozygous recessive mutations. Based on this hypothesis, variants were filtered based on a read depth>4 for quality and excluded synonymous variants. This resulted in 258 indels and 13,632 SNPs as candidate mutations. See FIG. 1.

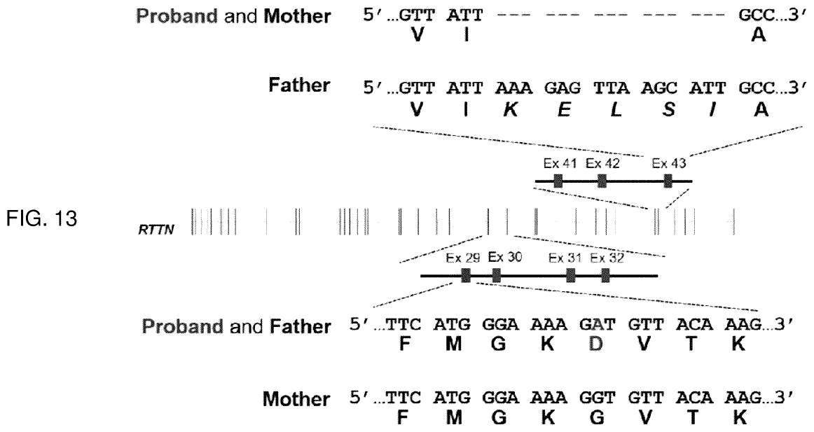

Next, variants that were not rare (minor allele frequencies>0.1%) and variants where both parents were heterozygous carriers or either parent was homozygous were excluded, resulting in 37 indels and 215 SNPs as candidates. Finally, a three-fold filter of functional prediction (exclusion of benign prediction using polyphen2), expression in CMs or heart tissue and validation by Sanger sequencing, yielded a single candidate gene, RTTN (the gene encoding rotatin). The RTTN mutation consisted of an in-frame deletion (removing amino acid residues p.1921-1925) inherited from the mother and a G1321D missense mutation inherited from the father. See FIG. 13, FIG. 1, and Table 4. Whole-exome sequencing of the proband and his parents revealed that the patient was a compound heterozygote for a G1321D missense mutation from the father and an in-frame deletion (p.1921-1925) from the mother.

Confirmation that the iPSCs were derived from the patient were heterozygotes were 50/50 was performed using Sanger Sequencing. To confirm RTTN as the causal gene, CRISPR/Cas9 technology was used to generate knock-out (KO) and gene-corrected (GC) iPSCs. See FIG. 6A for the general strategy for generation of RTTN KO and GC lines. The region corresponding to p.G1321 on exon 29 was targeted for KO in control iPSCs by nonhomologous end-joining (NHEJ), and the region containing the p.G1321D mutation in cDCM iPSC edited by homology-directed repair (HDR). For KO iPSCs, a CRISPR/Cas9 single-guide RNA (sgRNA) targeting a protospacer-adjacent motif (PAM) site was derived and placed N-terminal to the region corresponding to p.G1321D in exon 29 of RTTN. FIG. 6B shows single-guide RNA (sgRNA) targeting the PAM site (green) in RTTN exon 29. Using this sgRNA, four RTTN KO lines were generated from isogenic control iPSCs containing indels, which were then confirmed by Sanger sequencing. We generated four independent KO lines that were confirmed by Sanger sequencing. Dotted lines indicate deletions and red letters indicate insertions.

To generate GC iPSCs, another CRISPR/Cas9 sgRNA was designed close to the p.G1321D missense mutation (FIG. 6C) and the repair template to correct this mutation by homology-directed repair (FIG. 6D). sgRNA for genome correction in cDCM iPSCs targets the PAM site (green) near the region on exon 29 that corresponds to the p.G1321D mutation (violet). The repair templates contains the reference sequence at G4018 (violet), corresponding to p.G1321. The repair template also contained a “beacon” (red), which does not change the amino acid sequence, to confirm gene repair.

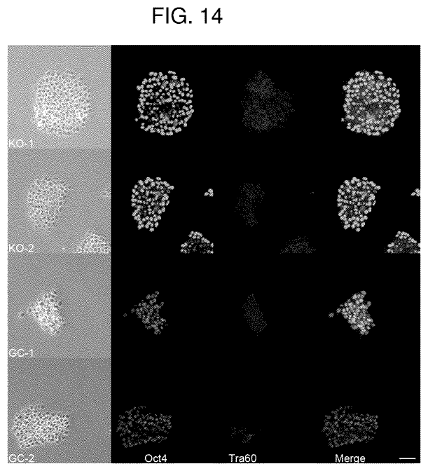

To ensure that the GC lines were indeed corrected, the repair template had a silent G→A mutation in the PAM site in order to prevent cleavage by Cas9 as a molecular signature for missense repair (FIG. 6D). Several gene-corrected (GC) isogenic lines were generated from the iDCM-1 iPSC line in which the variant adenine (A) at position n.3962 was corrected to the reference guanine (G) (FIG. 6E). A total of nine gene-corrected iPSC lines were generated and confirmed by Sanger sequencing. The KO and GC lines contained no alterations at any of the potential off-target sites (see Table 2 and Table 3), and each of the lines were confirmed for stemness (FIG. 14) and cardiomyogenic potential (FIG. 5).

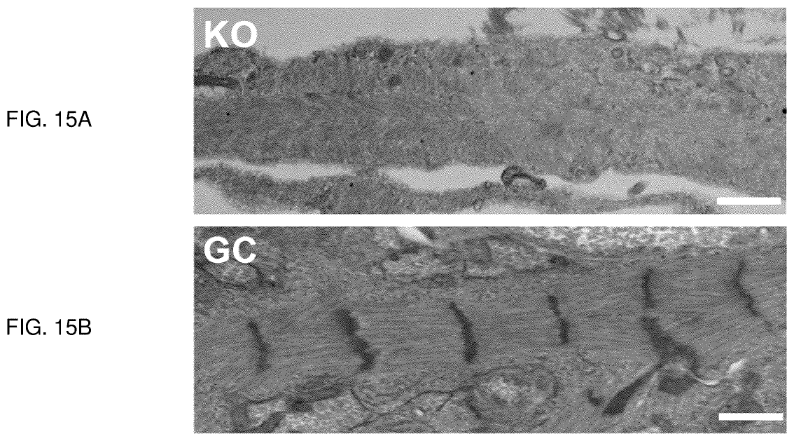

Transmission electron microscopy ultrastructural studies of KO and GC-CMs at day 45 of cardiac differentiation revealed disorganized sarcomere structures with severely disrupted Z-lines in the KO-CMs when compared to control-CMs. See FIG. 7. FIG. 15A and FIG. 15B show that RTTN knockout (KO) CMs displayed disorganized myofibrils with indistinct Z-lines (FIG. 15A), whereas the RTTN gene-corrected (GC) CMs (FIG. 15B) appear similar to the healthy control-CMs seen in FIG. 7. Scale bar=0.5 μm. By contrast, GC-CMs resembled the control-CMs, exhibiting well-organized sarcomere structures with intact Z-lines.

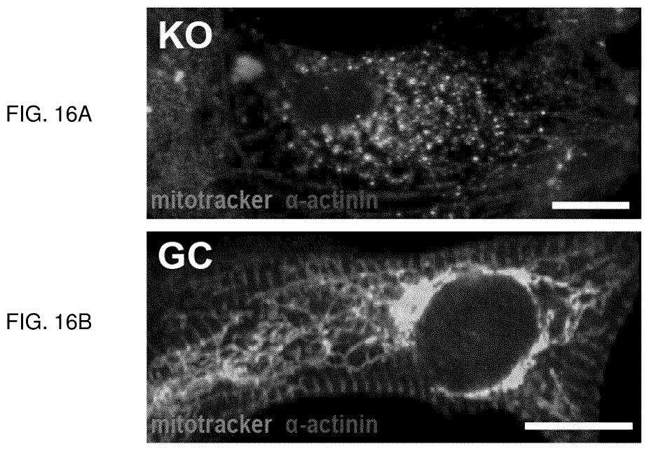

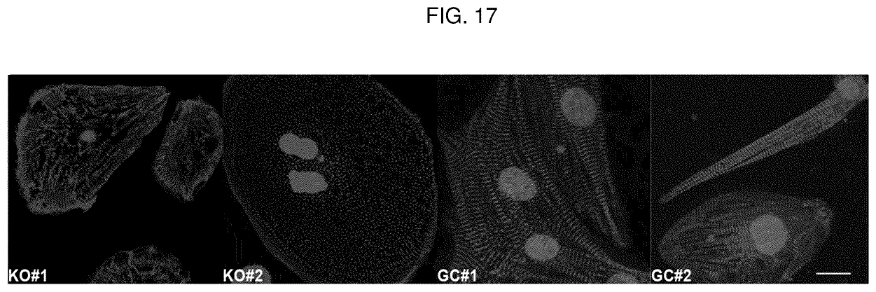

Immunofluorescence analysis revealed that the KO-CMs exhibited sarcomere disarray and disorganized Z-lines similar to the iDCM-CMs (FIG. 16A, FIG. 16B, FIG. 7E, FIG. 7F), whereas sarcomeres and Z-lines were largely restored in the GC-CMs, similar to the control-CMs. MitoTracker™ staining revealed dysmorphic, punctate mitochondria in the KO-CMs (FIG. 16A and FIG. 16B), similar to those observed in the iDCM-CMs. In contrast, the GC-CMs exhibited interwoven networks of elongated mitochondria, as observed in the control-CMs (FIG. 16A and FIG. 16B). RTTN KO-CMs (FIG. 16A) displayed globular or punctate mitochondria, as visualized by MitoTracker™ (green), and disorganized myofilaments, as visualized by α-actinin staining (red). In contrast, RTTN GC-CMs (FIG. 16B) displayed networks of elongated mitochondria (green) and organized myofilaments, similar to healthy control-CMs. Scale bar=20 μm. See also FIG. 17, which shows that normal sarcomere structure is restored in RTTN GC-iPSC-CMs compared with RTTN KO iPSC-CMs.

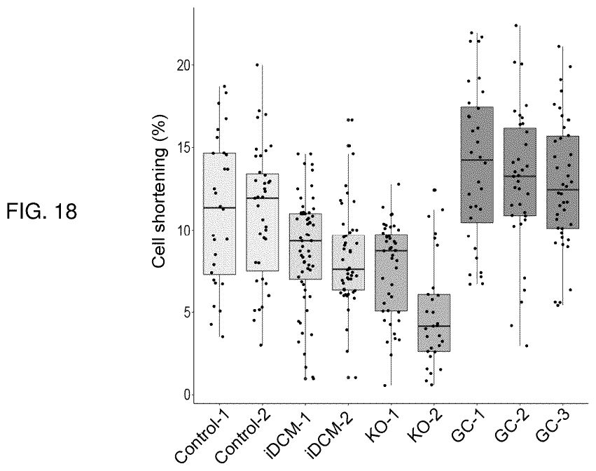

Importantly, KO-CMs from two independent KO iPSC lines exhibited weakened cell shortening (˜5-8%), similar to that observed in the iDCM-CMs (˜8%), as shown in FIG. 18. In contrast, cell shortening was restored in GC-CMs from three isogenic GC lines generated from the iDCM-1 iPSC line and was comparable to that of the control-CMs (n=28). Control 1-CMs, 39 Control 2-CMs, 59 iDCM 1-CMs, 48 iDCM 2-CMs, 43 KO 1-CMs, 29 KO 2-CMs, 32 GC 1-CMs, 35 GC 2-CMs. Center line=median; whiskers=1.5IQR.

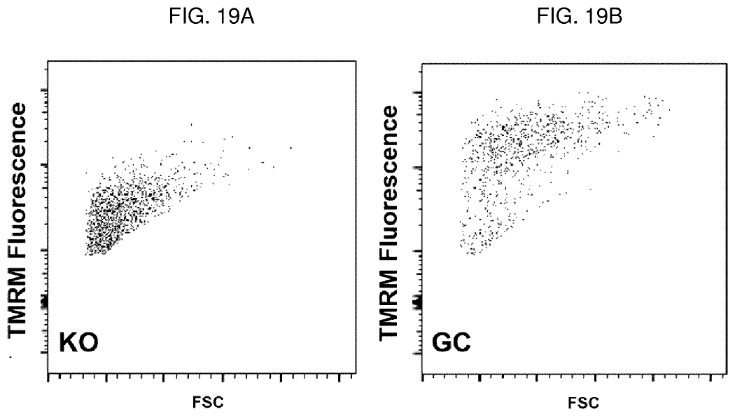

Moreover, TMRM staining indicated a marked reduction in the mitochondrial membrane potential in the KO-CMs, comparable to that observed in the iDCM-CMs, whereas the mitochondrial membrane potential was restored in the GC-CMs. FACS analysis of TMRM staining demonstrating reduced mitochondrial membrane potential in RTTN KO-CMs and restored mitochondrial membrane potential in RTTN GC-CMs. See FIG. 19A, FIG. 19B.

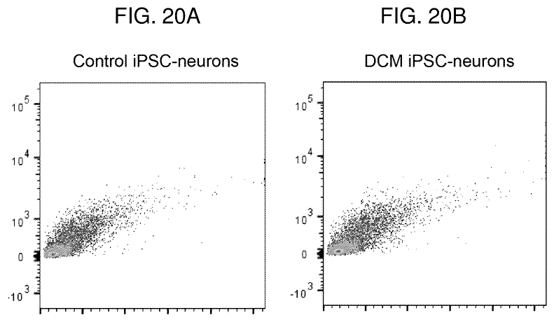

Additionally, the increased proliferation capacity observed in iDCM-CMs was seen in KO-CMs, while GC-CMs displayed normal levels of proliferation (FIG. 20). Taken together, these data demonstrate that RTTN is the causal gene for the sarcomere and mitochondrial defects observed in the iDCM patient.

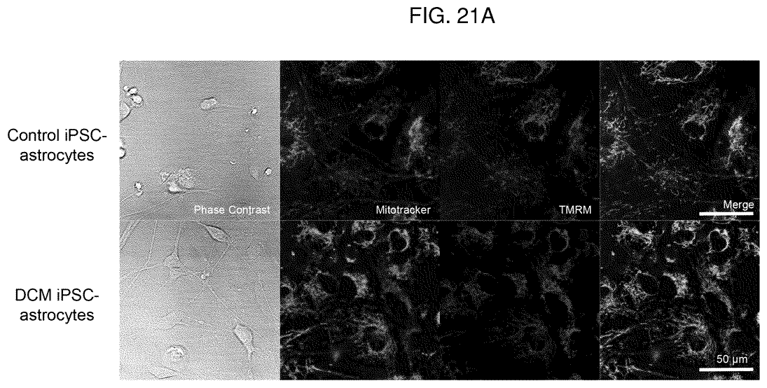

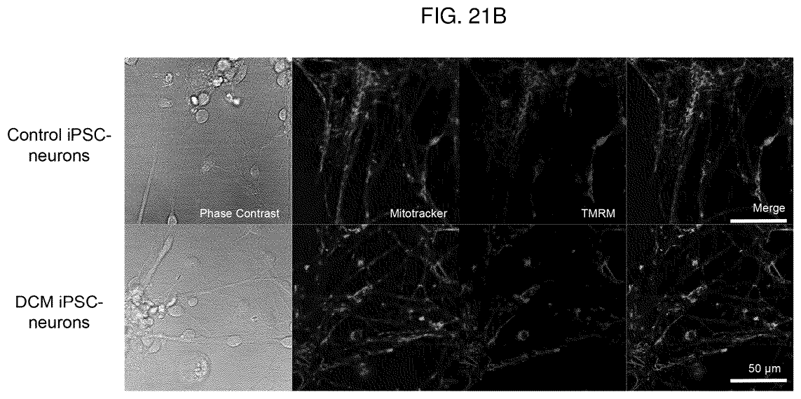

Previous studies found that a distinct set of homozygous recessive mutations in RTTN cause congenital brain defects including microcephaly. Whether there were defects in mitochondrial morphology and function in astrocytes and neurons derived from control and iDCM iPSCs was investigated, although the iDCM patient had no neurocognitive deficits. There were no apparent morphological or mitochondrial membrane potential differences between iDCM and control iPSC-derived astrocytes and neurons. See FIG. 21A, FIG. 21B, and FIG. 20, which show the mitochondrial structure and function of control and cDCM iPSC-derived astrocytes and neurons.

### Example 4: RTTN Mutation Leads to Dilated Cardiomyopathy in Multiple In Vivo Systems

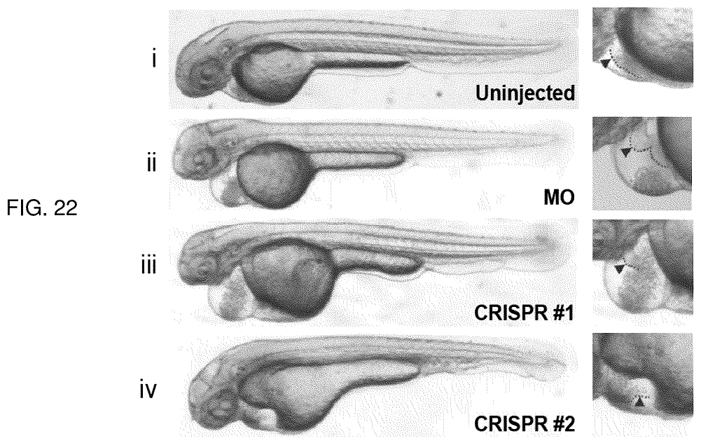

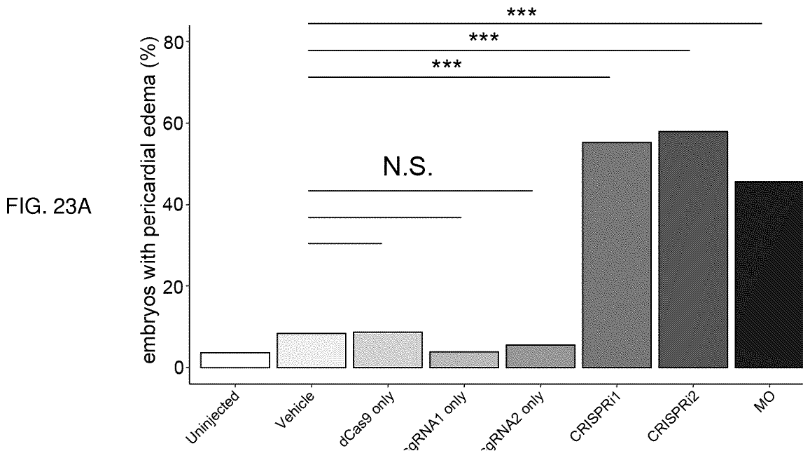

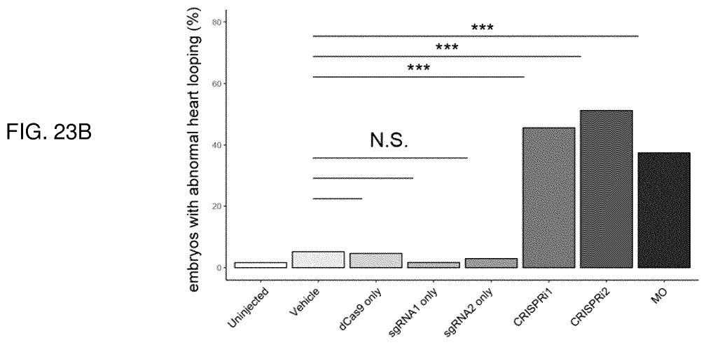

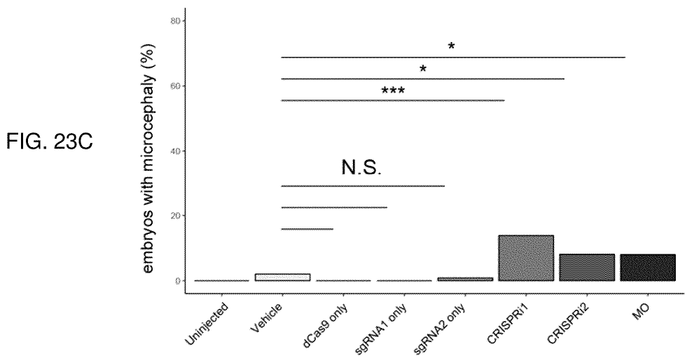

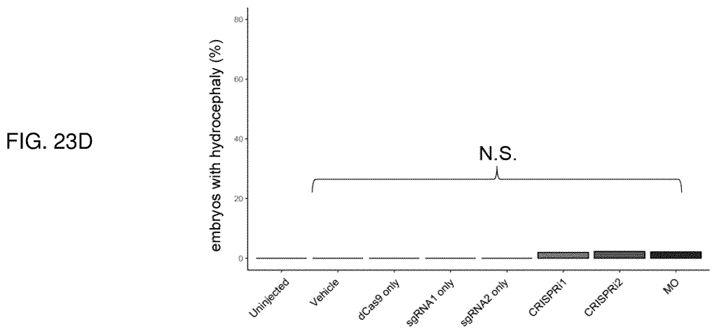

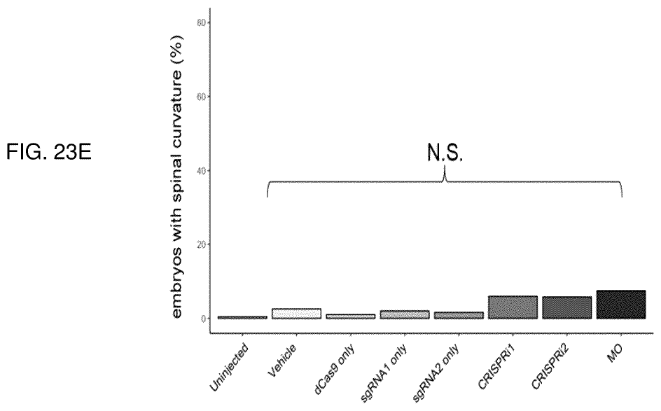

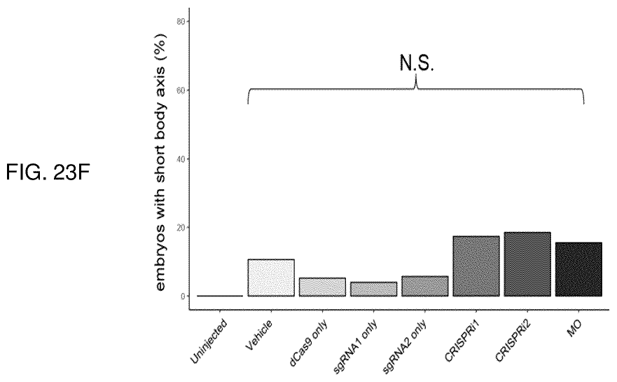

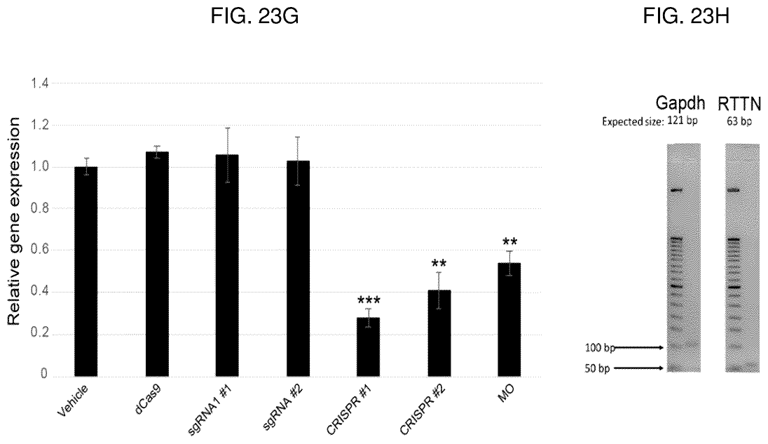

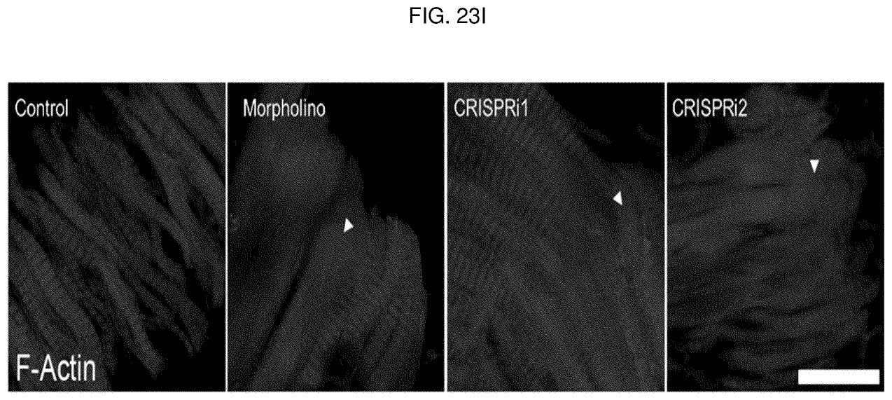

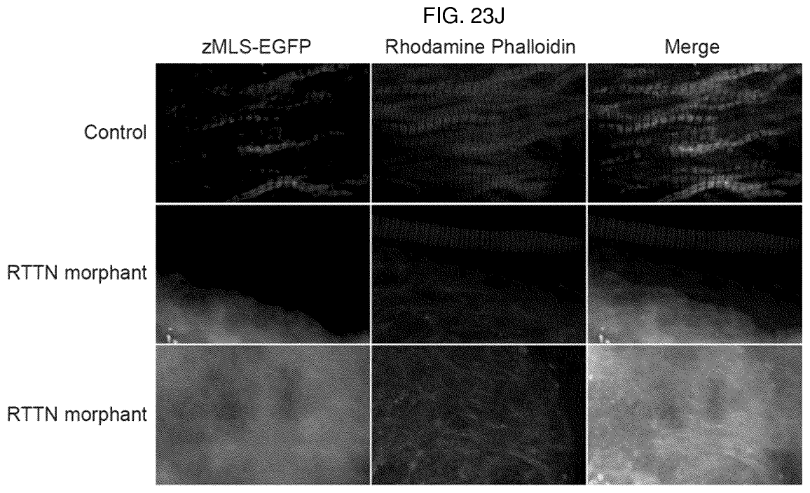

To test whether RTTN plays an evolutionarily conserved role in the heart through genetic knockdowns/knockouts in multiple in vivo systems, we first examined zebrafish embryos as they are particularly well suited for studying gene function during cardiovascular development. Following knockdown of RTTN in developing zebrafish embryos, through morpholino (MO) and CRISPRi, which inactivates target gene transcription, a significant percentage of embryos at 48 hours post-fertilization displayed a large pericardial edema (Vehicle=8.4%; CRISPRi1=55.3%, CRISPRi2=57.9%, MO=45.6%), a reliable marker of heart failure in zebrafish. See FIG. 22 and FIG. 23. FIG. 22 shows representative images of embryos at 48 hpf: (i) uninjected embryo; (ii) embryo injected with translational blocking morpholino (MO); embryos injected with CRISPRi #1 (iii) and #2 (iv). The enlarged images on the right of FIG. 22 shows corresponding enlarged images of the heart. Arrowhead, outline of heart. FIG. 23A shows the incidence of pericardial edema with impaired tail circulation in 48-hpf embryos. Results n≥101 embryos from n≥7 biological replicates using embryos from n≥2 breeding pairs. Vehicle was 0.08% phenol red+PBS in ultrapure water. *** p<0.00017 by Fisher's exact test with Bonferroni Correction; N.S.=not significant.

In addition, CRISPRi and MO-injected embryos exhibited abnormal developmental morphologies including abnormal heart looping (see FIG. 23B), in agreement with previous studies of global knockout of Rotatin in mice. A small but significant number of fish exhibited microcephaly (see FIG. 23C). However, there were no significant increases in the occurrence of hydrocephaly, spinal curvature, or short body axis (see FIG. 23D, FIG. 23E, and FIG. 23F).

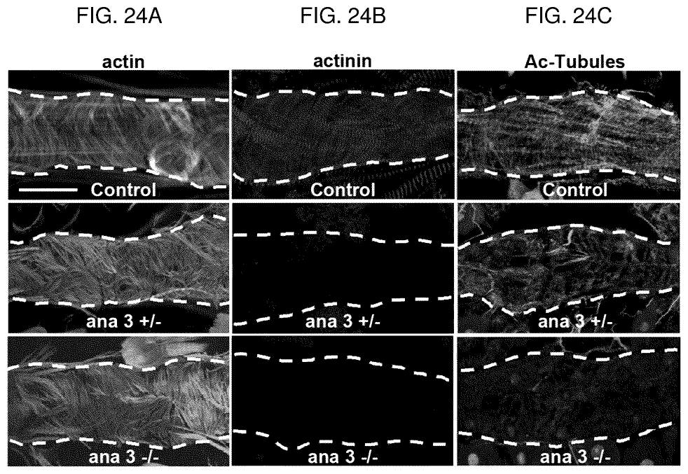

After verifying successful knockdown of RTTN in zebrafish embryos via RT-qPCR (FIG. 23G and FIG. 23H), we were then interested in if sarcomere structure was disrupted in developing zebrafish embryos. Upon staining with F-Actin, we saw regions of sarcomere malformation in knockdown embryos (both morpholino and CRISPRi), suggesting RTTN plays an important role in sarcomere development. See FIG. 23I. Use of a mitochondrial GFP tag in zebrafish suggested disruption of mitochondria in RTTN morphants, in line with our previous findings shown in FIG. 23J. FIG. 24A, FIG. 24B, and FIG. 24C show that knockdown of ana3 in Drosophila leads to impaired sarcomere and microtubule formation. The images show immunofluorescence staining actin, actinin, and acetylated (Ac) alpha-tubulin, respectively, in adult hearts (segment A4) from control (w1118), ana3^{+/-} and ana3^{-/-} flies. Scale bar=50 μm.

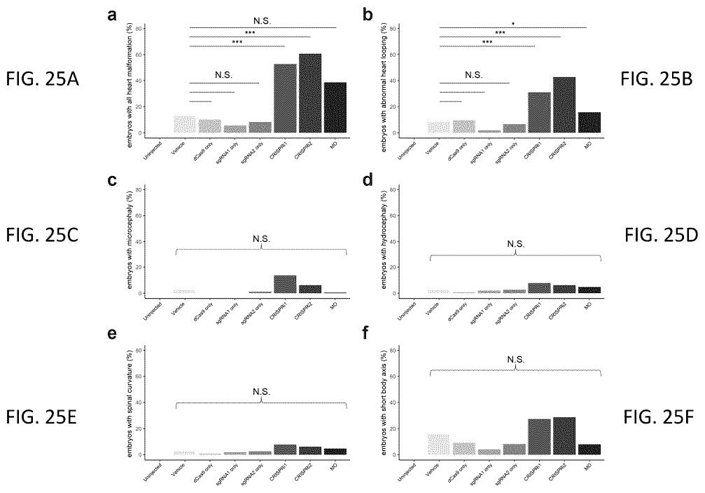

See the data in FIG. 25A through FIG. 25F for results from studies showing that RTTN knockdown phenotypes in zebrafish are consistent with ciliopathies. Refer to FIG. 25A and FIG. 25B for evidence that knockdown of RTTN resulted in pericardial edema, heart looping defects, and heart malformations. Refer to FIG. 25C through FIG. 25F for a report of the observations of higher incidences of hydrocephaly, microcephaly, abnormal spinal curvature, and severe anterior-posterior patterning defects. These trends did not reach significance in this study. Embryos were assessed at 48 hpf. n≥4 biological replicates using embryos from n≥2 breeding pairs. Vehicle was 0.08% phenol red+PBS in ultrapure water. Multiple comparisons were made between the vehicle and treatments using Fisher's exact test with Bonferroni's correction. * P<0.0085, *** P<0.00017, N.S.=not significant.

Due to the co-occurrence of heart looping and pericardial edema in zebrafish, we used the Drosophila system, which has been established as an excellent model in which to study dilated cardiomyopathy and does not have a looped heart tube, to determine more specifically whether RTTN is associated with dilated cardiomyopathy. The human RTTN gene has a highly conserved and unique fly homolog called ana3. We obtained ana3 mutant flies with P-element insertions 3 BPs upstream of the initiating ATG, resulting in a null phenotype. The ana3 mutant flies were viable but died quickly after eclosion.

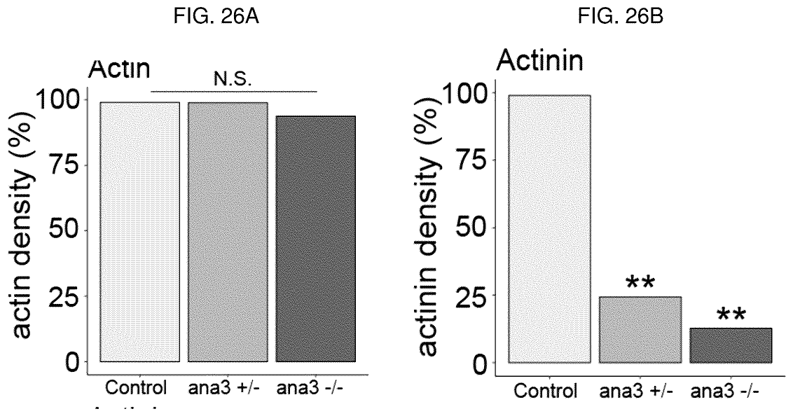

Quantitation of immunofluorescent imaging of actin and actinin in adult heart (segment A4) from control (w1118) was performed in ana3^{+/-} and ana3^{-/-} flies. Statistical results of normalized cardiac fiber density (N=10). * P<0.05; ** P<0.01. See FIG. 26A and FIG. 26B. This staining of structural actin with phalloidin determined that there was no significant difference between ana3 mutant and control embryos. See FIG. 26A, FIG. 26B, and FIG. 24A. Immunostaining for actinin, a protein localized normally in Z-lines, revealed that actinin structures were almost completely abolished (p<0.01) in both ana3 heterozygous and homozygous mutant Drosophila hearts. See FIG. 26A, FIG. 26B, and FIG. 24B. These findings demonstrate the importance of ana3 for heart structural development.

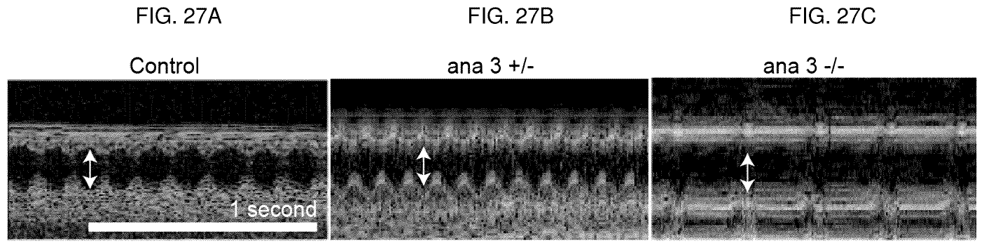

Given the severe structural deficits in cardiac filaments observed in the hearts of both ana3 mutants, we used optical coherence tomography to investigate whether these mutations affected cardiac contractile function. M-mode scans were selected at the maximum diastolic event for each genotype. Cross-sections of the heart tube obtained in this way showed a significantly increased end-diastolic dimension (EDD) in the ana3 homozygote fly hearts, but not in the ana3 heterozygote mutant hearts. FIG. 27A, FIG. 27B, and FIG. 27C are OCT images for heart of control, ana3+/− and ana3−/− flies. Arrowheads indicate\ the end-diastolic diameter (EDD).

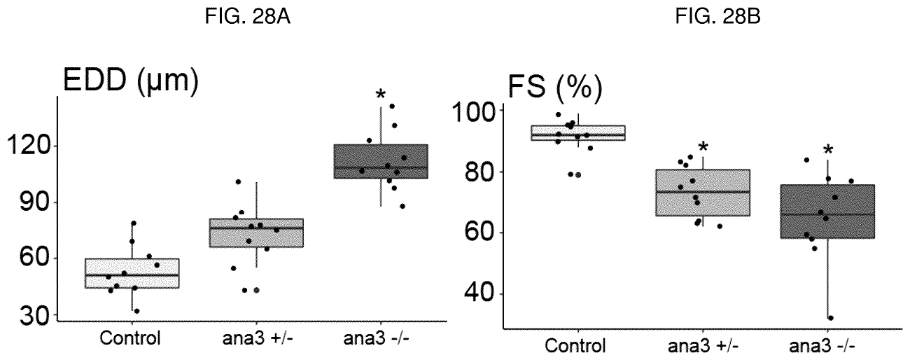

The M-mode orthogonal heart views provided precise and real-time measurements of the heart tube diameter and heart rate. Compared with control embryos, both ana3 heterozygous and homozygous mutant flies showed significantly reduced fractional shortening (FS). FIG. 28A and FIG. 28B shows results for statistical analysis for EDD (μm) and percent fractional shortening (FS) obtained from the optimal computed tomography data. Each datapoint represents the average of measurements from three heartbeats randomly selected within a two-second time frame for each fly. Center line=median; whiskers=1.5IQR for each genotype. * P<0.05.

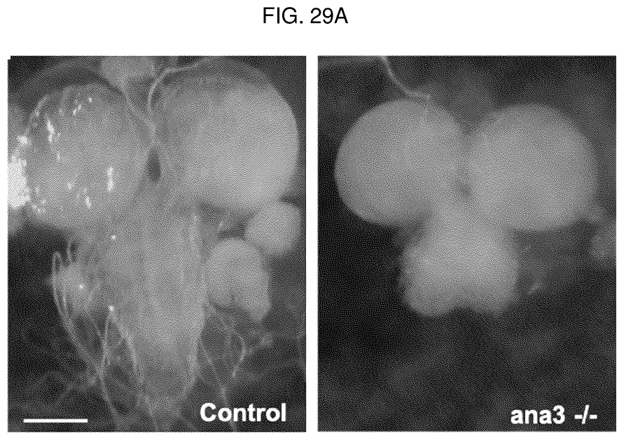

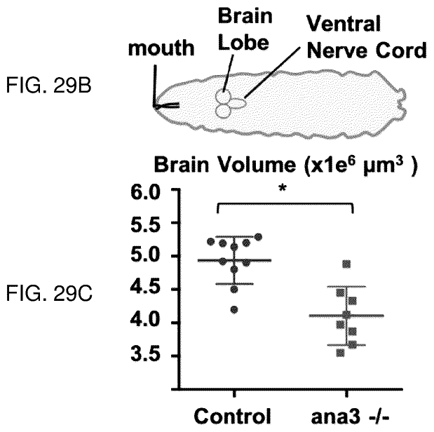

In addition to the heart defects in Drosophila, we also observed that the brain lobes of homozygous ana3 deficient third instar larvae appeared smaller than those of control larvae (see FIG. 29A and FIG. 29B), similar to the phenotype observed in patients with microcephaly due to RTTN variants. Statistical analysis confirmed that ana3 homozygous larvae had significantly reduced brain volume compared with WT larvae. FIG. 29C. The ana3 heterozygous larvae showed only a slightly early ventral nerve cord dissociation phenotype, suggesting that RTTN may have important functions in multiple organs. These data suggest evolutionarily conserved roles for RTTN in cardiac development, structure, and function.

### Example 5: Impaired Maturation of iDCM-CMs Revealed by scRNA-Seq

Based on the developmental age (3 months, perinatal) of the patient when the iDCM first presented and the broad set of phenotypic defects modeled in our iPSC-CM system, we suspected that CM maturation might be involved in the etiology of the iDCM. ScRNA-seq has emerged as an extremely powerful tool to study and analyze CM maturation dynamics. Thus, we designed a scRNA-seq study aimed at testing the hypothesis that CM maturation is affected in iDCM. We performed scRNA-seq on d35 control and iDCM-CMs using the 10× chromium platform. After quality control and filtering of non-CMs (TNNT2/MYH6-clusters) and cells undergoing the cell cycle, we began analysis of 4,671 control and 1,080 iDCM-CMs. We then performed differential gene testing of the control and iDCM cells, and found that 275 genes were significantly downregulated and 618 genes were significantly upregulated in the iDCM-CMs.

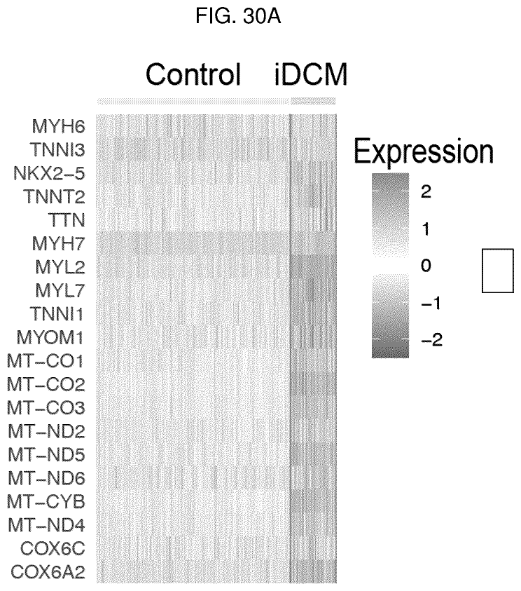

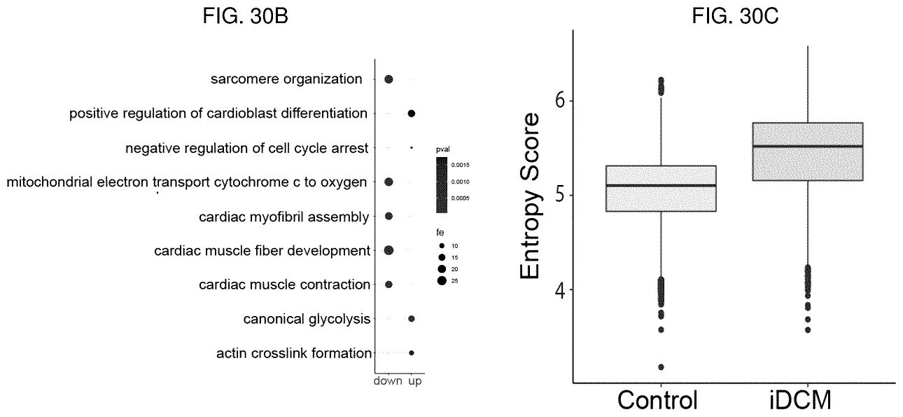

We began our analysis of these genes by investigating the expression of a broad set of genes previously shown to be associated with and important to CM maturation that were consistently downregulated in iDCM-CMs. FIG. 30 is a heat map showing expression of genes related to CM maturation in control and iDCM-CMs.

To determine the affected pathways, we then performed gene ontology (GO) enrichment analysis on the iDCM-CMs and found that terms related to processes essential for heart maturation and function, such as sarcomere organization, cardiac muscle contraction, electron transport-coupled proton transport, and cardiac myofibril assembly, among others, were downregulated in iDCM-CMs, whereas GO terms related to glycolysis, and apoptosis were enriched. See FIG. 30B, a dot plot of fold enrichment (fe) of gene ontology (GO) pathways. up=upregulated in iDCMs; down=downregulated in iDCMs.

A recently published maturation scoring metric, which provides an unbiased approach to determining the maturation status of CMs based on transcriptomic entropy of each CM then was used. The analysis showed an increase in transcriptomic entropy, corresponding to a decrease in maturity, of iDCM-CMs when compared with control-CMs. See FIG. 30C, a Shannon entropy score showing relative maturation score of control and iDCM-CMs. Taken together, these data support that CM maturation is impaired in the iDCM-CMs.

### Example 6: Defective Centrosomal Reduction in iDCM

During CM differentiation and maturation, the centrosome is known to undergo dramatic changes which include unpairing or “splitting” of the centriole, disappearance of certain pericentriolar proteins like CEP135, and the re-localization of other pericentriolar proteins, such as pericentrin (PCNT) and pericentriolar material 1 (PCM1), to form a MTOC at the nuclear envelope. The transition of PCNT during CM differentiation involves a key isoform switch (from PCNT-B to PCNT-S), which contributes to postnatal cell cycle arrest through the preferential expression of PCNT-S in the perinuclear centrosome.

Rotatin (encoded by RTTN) is known to be a centrosomal protein in both Drosophila (ana3) and humans, and has been previously shown to be involved in regulation of centrosome structure. Based on this and the observed CM maturation defect in the iDCM-CMs, we asked if developmentally programed centrosomal changes associated with CM maturation were disrupted in the iDCM-CMs.

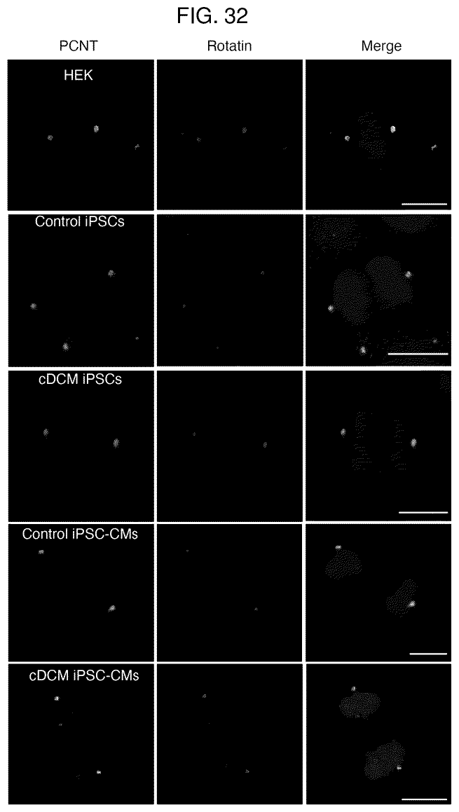

Although we could not localize rotatin to the centrosome using commercially available rotatin antibodies, we demonstrated its centriolar localization in HEK293 cells using SASY, a polyclonal antibody directed against the N-terminus of rotatin. See FIG. 32. Additionally, using SASY, we detected rotatin in the centrosomes of iPSCs and CMs actively in mitosis (FIG. 32G). Based on SASY immunostaining, there were no differences in rotatin localization between iDCM and control cells.

FIG. 32 is a set of photomicrographs showing that Rotatin (RTTN) is colocalized to the centrosomes in HEK293 cells, iPSCs, and iPSC-CMs. RTTN was detected using the SASY antibody, which is directed against the N-terminal region of RTTN. In HEK293 cells, SASY detected RTTN in both proliferating and quiescent cells; however, in iPSCs and iPSC-CMs, SASY detected RTTN predominantly in proliferating cells. The were no gross differences between RTTN localization in WT and cDCM iPSCs and iPSC-CMs, as detected by SASY.

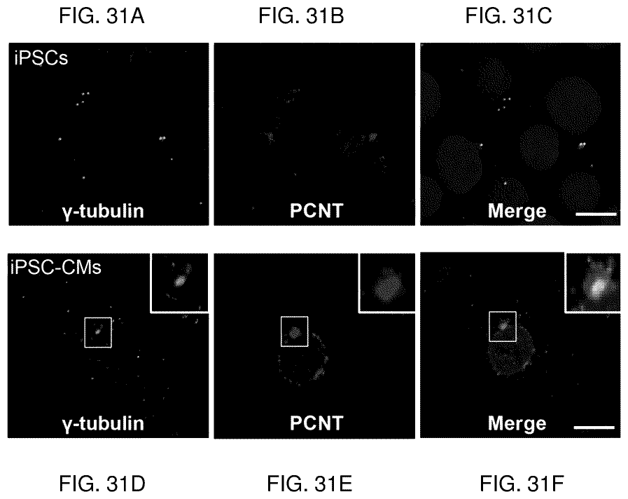

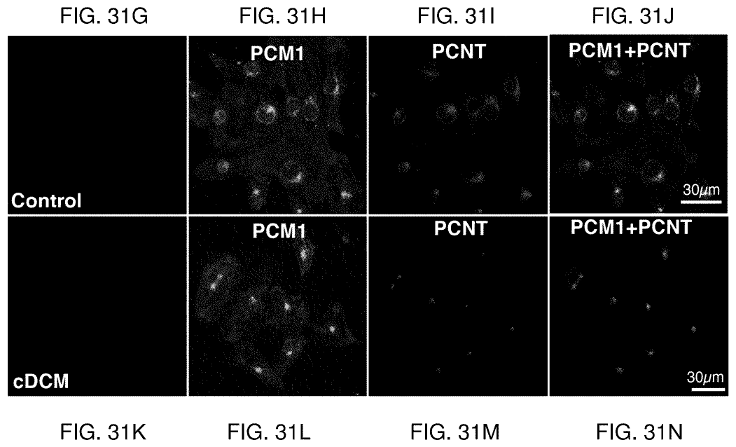

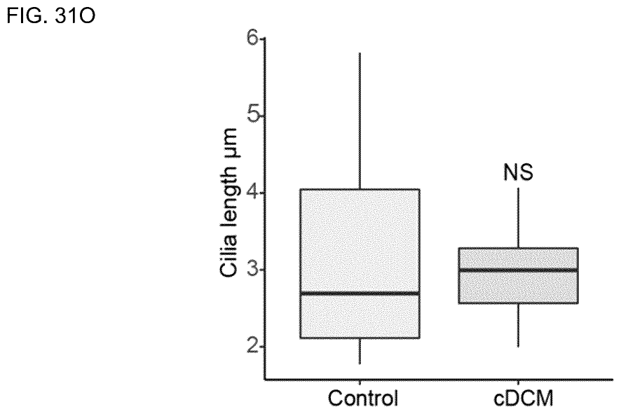

In control iPSCs, PCNT colocalizes with the centriolar component y-tubulin (FIG. 31A), but as iPSC-CMs mature, both PCNT and PCM1 become largely perinuclear (FIG. 33A and FIG. 33B), indicating that centrosome reduction. In contrast, in the iDCM-CMs, both PCNT and PCM1 localization remained mostly centriolar (FIG. 33A and FIG. 33B), suggesting that this process is impaired in RTTN mutant CMs. Importantly, there were not any significant changes in primary cilia length. See FIG. 31O. FIG. 33A shows that in the control CM with a normally organized sarcomere (α-actinin, red), PCNT (green) is redistributed to the perinuclear region (arrowheads). FIG. 33B shows that the RTTN mutant (iDCM) exhibits CM with a disorganized sarcomere (α-actinin, red), where PCNT (green) remains localized to the centrosome. Quantitation of PCNT distribution in D36 control and iDCM-CMs was performed by a blinded observer with categories of centriolar, split, or perinuclear. PCNT was perinuclear in 21.65% of control-CMs, whereas it was perinuclear in only 7.26% of iDCM-CMs. Perinuclear vs centriolar p=0.0021 by two-tailed Fisher's exact test. n=97 control-CMs, 124 iDCM-CMs.

See also FIG. 31H through FIG. 31N, which show the distribution of PCNT and PCM1 in iPSC-CMs.

Whether reorganization of the centrosome is affected in KO-CMs and recovered in GC-CMs was then investigated. Interestingly, there was a decrease in perinuclear localization of the centrosome in KO-CMs when compared to control-CMs (21.6% in control-CMs compared to 6.6% in KO-CMs) and an increase in perinuclear centrosome in GC-CMs when compared to iDCM-CMs (7.3% in iDCM-CMs compared to 27.7% in GC-CMs). See FIG. 33B.

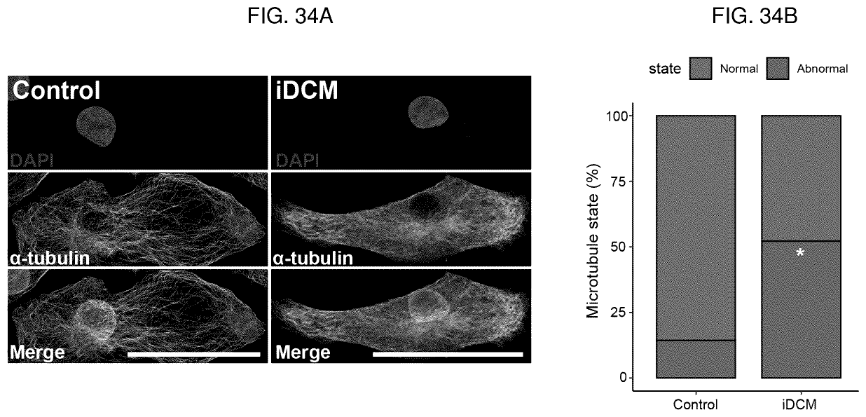

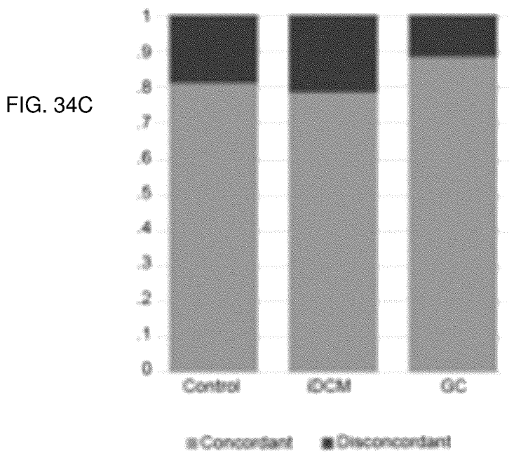

To determine if these changes in centrosome structure were linked to proper sarcomere development, the proportion of CMs that displayed concordant centrosome and sarcomere state (reduced centrosomes with mature sarcomeres and unreduced centrosomes with immature sarcomeres) was measured. There were significant levels of concordance in control, iDCM, and GC-CMs (see FIG. 34C), showing that the process of centrosome reduction is linked to proper formation.

Taken together, these results suggest that rotatin is essential for reorganization of the centrosome during cardiomyocyte development.

Following centrosomal changes in striated muscles, the nuclear envelope, which contains PCNT and PCM1, acts as the dominant microtubule organizing center (MTOC). Microtubules play a critical role in CMs, providing structural integrity, organizing sarcomere components, and serving as a mitochondrial highway system throughout the CM. Because or the defects observed in centrosomal reorganization in RTTN mutant CMs, we examined whether microtubule networks in iDCM-CMs were also disrupted. In iPSCs, we did not observe any gross differences in microtubule structures in control and iDCM-iPSCs immunostained for α-tubulin. See FIG. 34B. However, after cardiac differentiation, significant disruptions in the microtubule networks of the iDCM-CMs were frequently observed.

Instead of the prominent meshwork of thick microtubule fibers emanating from the perinuclear region observed in control-CMs (see FIG. 34A and FIG. 34B), the iDCM-CMs displayed much thinner, shorter, and less distinct microtubule fibers without a clearly organized center. FIG. 34A shows control CM displaying a prominent network of thick microtubule (MT) fibers (a-tubulin, green) emanating from the perinuclear region, with an organized sarcomere (cardiac TnT, red). FIG. 34B, in contrast, shows that iDCM CM displayed thinner, shorter and fainter MT fibers without a clear organizing center, and a disorganized sarcomere. Quantitation of CMs with grossly normal and abnormal MT network was performed by a blinded observer. The MT network was grossly normal in 86% of control-CMs, whereas only 48% of iDCM-CMs had normal MT networks (n=28). Control-CMs, 23 iDCM-CMs. p-0.006 by two-tailed Fisher's exact test. Scale bar=50 μm.

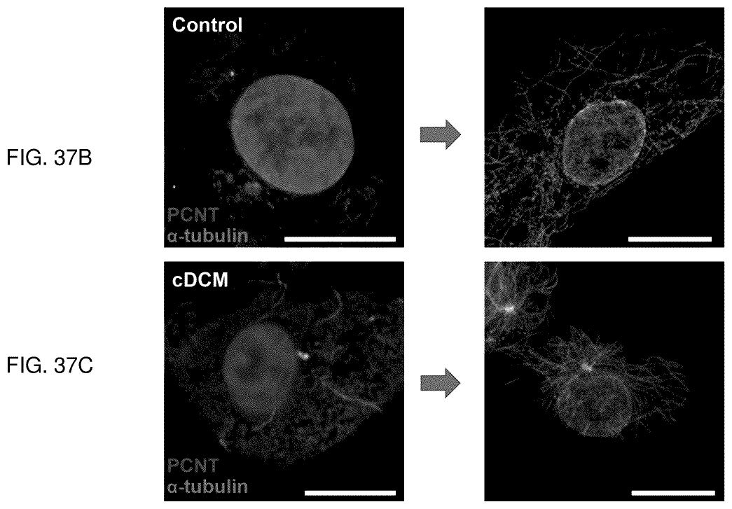

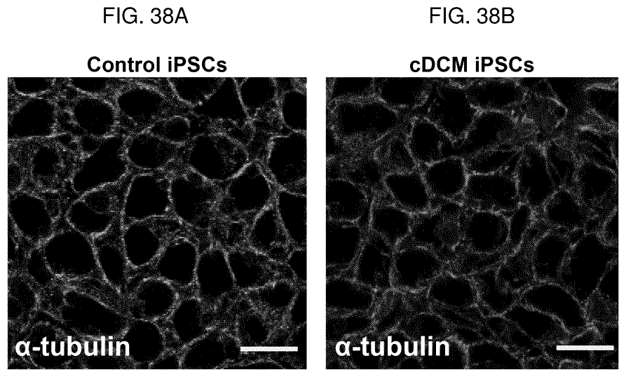

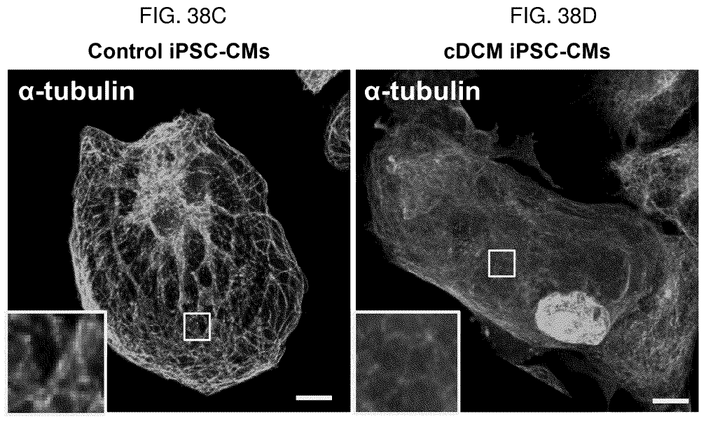

Defects in the microtubule networks of ana3 Drosophila mutant hearts also were observed. Interestingly, the tubulin assembly pattern revealed by immunostaining for acetylated alpha-tubulin (Ac-tubulin) was partially lost in ana3^{+/-} fly hearts and was severely disrupted in ana3^{-/-} fly hearts compared with control fly hearts (see FIG. 24C). These results support the hypothesis that mutations in RTTN affect CM structure and function through impaired centrosome reduction and consequent disruption of the microtubule network. FIG. 37A. In addition, microtubule regrowth assays showed that iDCM-CMs retain the ability to regrow microtubule networks after cold mediated disassembly. FIG. 37B and FIG. 37C.

### Example 7: Small-Molecule Treatment Rescues Impaired Maturation-Related iDCM in the IPSC Model

Because there currently are no therapeutic agents specifically for iDCM, whether ameliorating the CM maturation defect would be sufficient to rescue the structural and functional defects present in iDCM was tested. See the schematic in FIG. 35, which details C19 treatment at differentiation day 30 (d30) control-CMs and iDCM-CMs. Developmentally programmed centrosome changes are impaired in iDCM-CMs, so we treated them with a small molecule, C19, that previously has been shown to induce centrosome reduction in CMs.

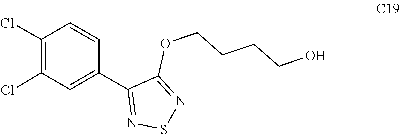 [SMILES: OCCCCOc1nsnc1-c1ccc(Cl)c(Cl)c1]

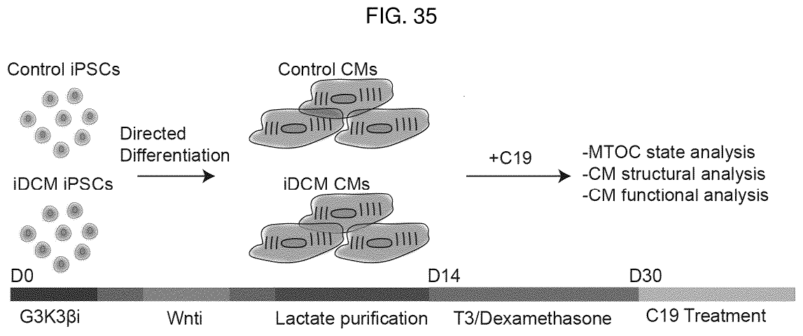

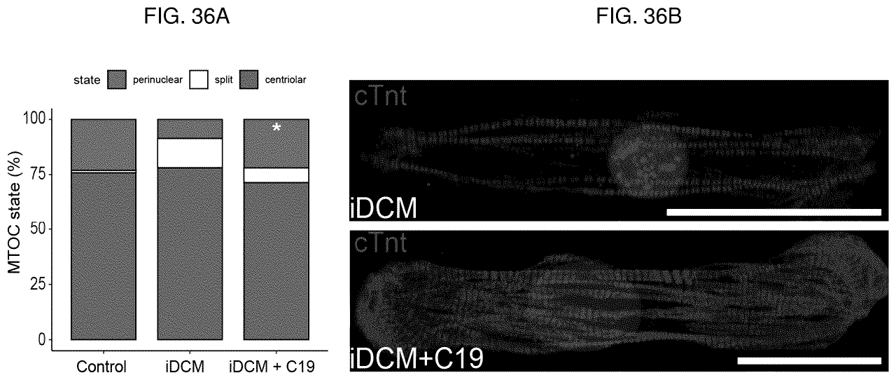

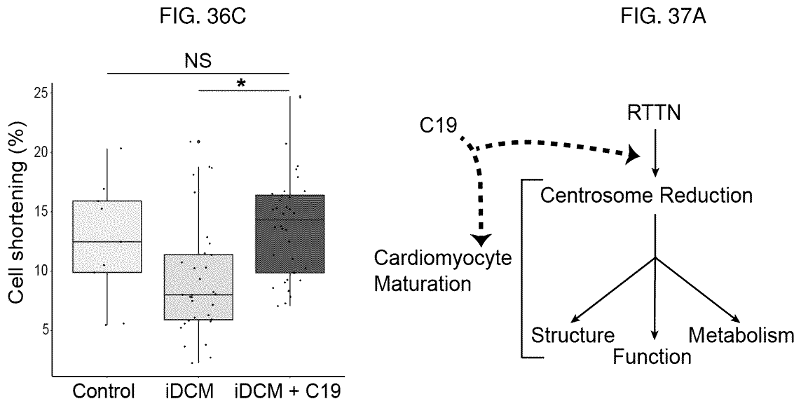

Interestingly, treatment with C19 facilitated improved initiation of the perinuclear MTOC in iDCM-CMs. FIG. 36A presents data on the quantitation of centriolar, split, and perinuclear MTOC localization in D32 CMs by a blinded observer. PCNT was perinuclear in 23.1% of control-CMs, 7.6% of iDCM-CMs, and 23.6% of iDCM+C19 CMs. p=0.0082 by Fisher Exact test. Moreover, the improved sarcomere formation (FIG. 36B and FIG. 36C) in the C19-treated iDCM-CMs demonstrated that structural aspects of CM maturation were significantly improved. FIG. 36B and FIG. 36C show representative images of untreated iDCM-CMs and iDCM-CMs treated with C19, respectively, showing increased levels of sarcomere formation. Scale bar=50 μm.

Importantly, contractility, a functional metric of CM maturation, was significantly improved in C19-treated iDCM-CMs (see FIG. 36C), with comparable levels of cell shortening in the treated iDCM-CMs and in healthy control-CMs. Boxplots showing % cell shortening in control, D40 iDCM, and iDCM C19 treated CMs. n=9 control, 32 iDCM, 36 iDCM+C19 from two differentiations. p value=0.0006 by one-way ANOVA. Post hoc Tukey correction p value of iDCM vs iDCM+C19=0.001, control vs iDCM+C19=0.682 (NS). Center line=median; whiskers=1.5IQR.

These results suggest that developmental centrosomal reorganizations are necessary for functional aspects of CM maturation to occur. See FIG. 37A, a working model of the CM maturation cascade showing RTTN-mediated centrosome reduction is upstream of other canonical maturation events. Facilitation of this process seems to be sufficient, according to results here, to rescue CM defects in this particular case of centrosome-mediated iDCM. This opens the door for pharmacological treatments for iDCM.

### Example 8: Treatment of Cardiomyopathy

For treatment of cardiomyopathy according to the invention, the practitioner preferably rules out myocarditis by heart biopsy, and follows with treatment by administering C19, or another YAP-TEAD (Hippo) pathway and/or a Wnt pathway inhibitor to a subject in need. Dose, route and timing of administration depends on the in vivo pharmacokinetic characteristics of each compound and the age and condition of the patient. Practitioners are able to determine the appropriate doses and dosage regimens for any particular patient. In general, however, about 1 mg/kg to about 100 mg/kg is an appropriate dose, given once daily to 4 times daily by intravenous injection, subcutaneous injection, by continuous infusion, or per os, necessary to maintain therapeutic concentrations, above the EC50 for each compound. The duration of therapy generally is about 1 to 14 days, depending on therapeutic response.

### REFERENCES

All references listed below and throughout the specification are hereby incorporated by reference in their entirety.
1. Agarwal, R. et al. Filamin C Cardiomyopathy Variants Cause Protein and Lysosome Accumulation. Circ. Res. (2021) doi: 10.1161/CIRCRESAHA.120.317076.
2. Alazami, A. M. et al. RTTN Mutations Cause Primary Microcephaly and Primordial Dwarfism in Humans. Am. J. Hum. Genet. 97, 862-868 (2015).
3. Ali, H., Braga, L. & Giacca, M. Cardiac regeneration and remodelling of the cardiomyocyte cytoarchitecture. FEBS Journal vol. 287 417-438 (2020).
4. Ashburner et al., “Gene ontology: Tool for the unification of biology,” Nature Genetics, vol. 25, no. 1. NIH Public Access, pp. 25-29, May-2000, doi: 10.1038/75556.
5. Basu D, Lettan R, Damodaran K, Strellec S, Reyes-Mugica M, Rebbaa A. Identification, Mechanism of Action, and Antitumor Activity of a Small Molecule Inhibitor of Hippo, TGF-b, and Wnt Signaling Pathways. Mol Cancer Ther 2014; 13. doi: 10.1158/1535-7163.MCT-13-0918.
6. Becker, R., Leone, M. & Engel. F. B. Microtubule Organization in Striated Muscle Cells. Cells vol. 9 1395 (2020).
7. Butler, P. Hoffman, P. Smibert, E. Papalexi, and R. Satija, “Integrating single-cell transcriptomic data across different conditions, technologies, and species,” Nat. Biotechnol., vol. 36, no. 5, pp. 411-420, June 2018, doi: 10.1038/nbt.4096.
8. Cadar A G, Feaster T K, Bersell K R, Wang L, Hong T T, Balsamo J A, et al. Real-time visualization of titin dynamics reveals extensive reversible photobleaching in human induced pluripotent stem cell-derived cardiomyocytes. Am J Physiol-Cell Physiol 2020; 318: C163-C173. doi: 10.1152/ajpcell.00107.2019.
9. Cavallin, M. et al. Recurrent RTTN mutation leading to severe microcephaly, polymicrogyria and growth restriction. Eur. J. Med. Genet. 61, 755-758 (2018).
10. Chen, C. Y. et al. Suppression of detyrosinated microtubules improves cardiomyocyte function in human heart failure. Nat. Med. 24, 1225-1233 (2018).
11. Chen, H. Y. et al. Human microcephaly protein RTIN interacts with STIL and is required to build full-length centrioles. Nat. Commun. 8, 1-14 (2017).
12. Chun, Y. W. et al. Combinatorial polymer matrices enhance in vitro maturation of human induced pluripotent stem cell-derived cardiomyocytes. Biomaterials 67, 52-64 (2015).
13. Cong and F. Zhang, “Genome engineering using CRISPR-Cas9 system,” Methods Mol. Biol., vol. 1239, pp. 197-217, 2015, doi: 10.1007/978-1-4939-1862-1_10.
14. Daniels et al., “A simple method for analyzing microsatellite allele image patterns generated from DNA pools and its application to allelic association studies,” Am. J. Hum. Genet., vol. 62, no. 5, pp. 1189-1197, 1998, doi: 10.1086/301816.
15. Depristo et al., “A framework for variation discovery and genotyping using next-generation DNA sequencing data,” Nat. Genet., vol. 43, no. 5, pp. 491-501, May 2011, doi: 10.1038/ng.806.
16. Faisst A M, Alvarez-Bolado G, Treichel D, Gruss P. Rotatin is a novel gene required for axial rotation and left-right specification in mouse embryos. Mech Dev 2002; 113:15-28. doi: 10.1016/S0925-4773 (02) 00003-5.
17. Feaster, T. K. et al. Matrigel Mattress: A Method for the Generation of Single Contracting Human-Induced Pluripotent Stem Cell-Derived Cardiomyocytes. Gire. Res. 117, 995-1000 (2015).
18. Feaster T K, Cadar A G, Wang L, Williams C H, Chun Y W, Hempel J E, et al. Matrigel Mattress: A Method for the Generation of Single Contracting Human-Induced Pluripotent Stem Cell-Derived Cardiomyocytes. Circ Res 2015; 117:995-1000. doi: 10.1161/CIRCRESAHA.115.307580.
19. Gene Ontology Consortium, “The Gene Ontology Resource: 20 years and still GOing strong,” Nucleic Acids Res., vol. 47, 2019, doi: 10.1093/nar/gky1055.
20. Gilbert L A, Larson M H, Morsut L, Liu Z, Brar G A, Torres S E, et al. CRISPR-Mediated Modular RNA-Guided Regulation of Transcription in Eukaryotes. Cell 2013; 154:442. doi: 10.1016/J.CELL.2013.06.044.
21. Grandone A, Torella A, Santoro C, Giugliano T, del Vecchio Blanco F, Mutarelli M, et al. Expanding the phenotype of RTTN variations: a new family with primary microcephaly, severe growth failure, brain malformations and dermatitis. Clin Genet 2016; 90:445-450. doi: 10.1111/cge. 12771.
22. Guo, F. Ye, Q. Sheng, T. Clark, and D. C. Samuels, “Three-stage quality control strategies for DNA re-sequencing data,” Brief. Bioinform., vol. 15, no. 6, pp. 879-889, August 2013, doi: 10.1093/bib/bbt069.
23. Guo et al., “Multi-perspective quality control of Illumina exome sequencing data using QC3,” Genomics, vol. 103, no. 5-6, pp. 323-328, 2014, doi: 10.1016/j.ygeno.2014.03.006.
24. Hafemeister and R. Satija, “Normalization and variance stabilization of single-cell RNA-seq data using regularized negative binomial regression,” Genome Biol., vol. 20, no. 1, p. 296, December 2019, doi: 10.1186/s13059-019-1874-1.
25. Han, P. Yi, X. Li, and E. N. Olson, “Hand, an evolutionarily conserved bHLH transcription factor required for Drosophila cardiogenesis and hematopoiesis,” Development, vol. 133, no. 6, pp. 1175-1182 Mar. 2006, doi: 10.1242/dev.02285.
26. Han and E. N. Olson, “Hand is a direct target of Tinman and GATA factors during Drosophila cardiogenesis and hematopoiesis,” Development, vol. 132, no. 15, pp. 3525-3536 Aug. 2005, doi: 10.1242/dev.01899.
27. Hattori et al., “Nongenetic method for purifying stem cell-derived cardiomyocytes,” Nat. Methods, vol. 7, no. 1, pp. 61-66, January 2010, doi: 10.1038/nmeth.1403.
28. Herkert J C, Abbott K M, Birnie E, Meems-Veldhuis M T, Boven L G, Benjamins M, et al. Toward an effective exome-based genetic testing strategy in pediatric dilated cardiomyopathy. Genet Med 2018; 20:1374-1386. doi: 10.1038/GIM.2018.9.
29. Joshi, P. et al. Huntington's disease associated resistance to Mn neurotoxicity is neurodevelopmental stage and neuronal lineage dependent. Neurotoxicology 75, 148-157 (2Q19).
30. Jumper, J. et al. Highly accurate protein structure prediction with AlphaFold. Nature 596, 583-589 (2021).
31. Kannan S, Farid M, Lin B L, Miyamoto M, Kwon C. Transcriptomic entropy benchmarks stem cell-derived cardiomyocyte maturation against endogenous tissue at single cell level. PLOS Comput Biol 2021; 17: e1009305. doi: 10.1371/JOURNAL.PCBI.1009305.
32. Khan R S, Pahl E, Dellefave-Castillo L, Rychlik K, Ing A, Yap K L, et al. Genotype and Cardiac Outcomes in Pediatric Dilated Cardiomyopathy. J Am Heart Assoc 2022; 11. doi: 10.1161/JAHA.121.022854/FORMAT/EPUB.
33. Kia, S. K. et al. RTTN mutations link primary cilia function to organization of the human cerebral cortex. Am. J. Hum. Genet. 91, 533-540 (2012).
34. Kimmel, C. B., Ballard, W. W., Kimmel, S. R., Ullmann, B. & Schilling, T. F. Stages of embryonic development of the zebrafish. Dev. Dyn. 203, 253-310 (1995).
35. Knight, W. E. et al. Maturation of Pluripotent Stem Cell-Derived Cardiomyocytes Enables Modeling of Human Hypertrophic Cardiomyopathy. Stem Cell Reports 16, 519-533 (2021).
36. Laustsen, P. G. et al. Essential Role of Insulin and Insulin-Like Growth Factor 1 Receptor Signaling in Cardiac Development and Function. Mol. Cell. Biol. 27, 1649-1664 (2007).
37. Lee T m, Hsu D T, Kantor P, Towbin J A, Ware S M, Colan S D, et al. Pediatr. Cardiomyopathyirc Res 2017; 121:855-873. doi: 10.1161/CIRCRESAHA.116.309386/FORMAT/EPUB.
38. Li et al., “Single cell expression analysis reveals anatomical and cell cycle-dependent transcriptional shifts during heart development,” Dev., vol. 146, no. 12, June 2019, doi: 10.1242/dev.173476.
39. Li and R. Durbin, “Fast and accurate short read alignment with Burrows-Wheeler transform,” Bioinformatics, vol. 25, no. 14, pp. 1754-1760 Jul. 2009, doi: 10.1093/bioinformatics/btp324.
40. Lian et al., “Directed cardiomyocyte differentiation from human pluripotent stem cells by modulating Wnt/β-catenin signaling under fully defined conditions,” Nat. Protoc., vol. 8, no. 1, pp. 162-175, December 2012, doi: 10.1038/nprot.2012.150.
41. Lipshultz S E, Law Y M, Asante-Korang A, Austin E D, Dipchand A I, Everitt M D, et al. Cardiomyopathy in Children: Classification and Diagnosis: A Scientific Statement From the American Heart Association. Circulation 2019; 140: E9-E68. doi: 10.1161/CIR.0000000000000682/FORMAT/EPUB.
42. Long P A, Evans J M, Olson T M. Diagnostic Yield of Whole Exome Sequencing in Pediatric Dilated Cardiomyopathy. J Cardiovasc Dev Dis 2017; 4. doi: 10.3390/JCDD4030011.
43. Madeddu, P. Defective Cardiomyocyte Maturation in the Congenitally Hypoplastic Right Ventricle: A Wrong Molecular Trajectory Leading to Heart Failure? J. Am. Heart Assoc. 9, 19433 (2020).
44. Mehdiabadi N R, Choon B, Sim B, Phipson B, Kalathur R K R, Sun Y, et al. Defining the Fetal Gene Program at Single-Cell Resolution in Pediatric Dilated Cardiomyopathy. Circulation 2022; 146:1105-1108. doi: 10.1161/CIRCULATIONAHA.121.057763.
45. Miyamoto M, Nam L, Kannan S, Kwon C. Heart organoids and tissue models for modeling development and disease. Semin Cell Dev Biol 2021. doi: 10.1016/j.semcdb.2021.03.011.
46. Monici, “Cell and tissue autofluorescence research and diagnostic applications,” Biotechnology Annual Review, vol. 11, no. SUPPL. Biotechnol Annu Rev, pp. 227-256, Sep-2005, doi: 10.1016/S1387-2656 (05) 11007-2.
47. Murayama, A. & Lechler, T. Microtubule organization, dynamics and functions in differentiated cells. Development (Cambridge) vol. 144 3012-3021 (2017).
48. Ng, D. C. H. et al. Centrosome Reduction Promotes Terminal Differentiation of Human Cardiomyocytes. Stem Cell Reports 15, 817-826 (2020).
49. Paige, S. L. et al. Patient-Specific Induced Pluripotent Stem Cells Implicate Intrinsic Impaired Contractility in Hypoplastic Left Heart Syndrome. Circulation 142, 1605-1608 (2020).
50. Ran, P. D. Hsu, J. Wright, V. Agarwala, D. A. Scott, and F. Zhang, “Genome engineering using the CRISPR-Cas9 system,” Nat. Protoc., vol. 8, no. 11, pp. 2281-2308, 2013, doi: 10.1038/nprot.2013.143.
51. Shen J, Zhang J H, Xiao H, Wu J M, He K M, Lv Z Z, et al. Mitochondria are transported along microtubules in membrane nanotubes to rescue distressed cardiomyocytes from apoptosis article. Cell Death Dis 2018; 9. doi: 10.1038/s41419-017-0145-x.
52. Shenje L T, Andersen P, Halushka M K, Lui C, Fernandez L, Collin G B, et al. Mutations in Alström protein impair terminal differentiation of cardiomyocytes. Nat Commun 2014 51 2014; 5:1-11. doi: 10.1038/ncomms4416.
53. Singh, D. P. & Patel, H. Left Ventricular Non-compaction Cardiomyopathy. Hear. Fail. Child Young Adult From Bench to Bedside 269-290 (2021).
54. Schindelin et al., “Fiji: An open-source platform for biological-image analysis,” Nature Methods, vol. 9, no. 7. Nat Methods, pp. 676-682, Jul-2012, doi: 10.1038/nmeth.2019.
55. Steinfeldt, J., Becker, R., Vergarajauregui, S. & Engel, F. B. Alternative splicing of pericentrin contributes to cell cycle control in cardiomyocytes. bioRxiv (2021) doi: 10.1101/2021.04.19.440474.
56. Steinfeldt J, Becker R, Vergarajauregui S, Engel F B. Alternative Splicing of Pericentrin Contributes to Cell Cycle Control in Cardiomyocytes. J Cardiovasc Dev Dis 2021, Vol 8, Page 87 2021; 8:87. doi: 10.3390/JCDD8080087.
57. Stevens, N. R., Dobbelaere, J., Wainman, A., Gergely, F. & Raff, J. W. Ana3 is a conserved protein required for the structural integrity of centrioles and basal bodies. J. Cell Biol. 187, 355-363 (2009).
58. Stouffs K, Moortgat S, Vanderhasselt T, Vandervore L, Dica A, Mathot M, et al. Biallelic mutations in RTTN are associated with microcephaly, short stature and a wide range of brain malformations. Eur J Med Genet 2018; 61:733-737. doi: 10.1016/j.ejmg.2018.06.001.
59. Stuart et al., “Comprehensive Integration of Single-Cell Data,” Cell, vol. 177, no. 7, pp. 1888-1902.e21, June 2019, doi: 10.1016/j.cell.2019.05.031.
60. Sydor A M, Coyaud E, Rovelli C, Laurent E, Liu H, Raught B, et al. PPP1R35 is a novel centrosomal protein that regulates centriole length in concert with the microcephaly protein RTTN. Elife 2018; 7. doi: 10.7554/eLife.37846.
61. Tidball A M, Neely M D, Chamberlin R, Aboud A A, Kumar K K, Han B, et al. Genomic instability associated with p53 knockdown in the generation of Huntington's disease human induced pluripotent stem cells. PLOS One 2016; 11. doi: 10.1371/journal.pone.0150372.
62. van der Meulen M H, Herkert J C, den Boer S L, du Marchie Sarvaas G J, Blom N, ten Harkel A D J, et al. Genetic Evaluation of A Nation-Wide Dutch Pediatric DCM Cohort: The Use of Genetic Testing in Risk Stratification. Circ Genomic Precis Med 2022. doi: 10.1161/CIRCGEN.120.002981
63. Vandervore, L. V. et al. Heterogeneous clinical phenotypes and cerebral malformations reflected by rotatin cellular dynamics. Brain 142, 867-884 (2019).
64. Vasilescu C, Ojala T H, Brilhante V, Ojanen S, Hinterding H M, Palin E, et al. Genetic Basis of Severe Childhood-Onset Cardiomyopathies. J Am Coll Cardiol 2018; 72:2324-2338. doi: 10.1016/J.JACC.2018.08.2171.
65. Viswanathan M C, Kaushik G, Engler A J, Lehman W, Cammarato A. A Drosophila melanogaster model of diastolic dysfunction and cardiomyopathy based on impaired troponin-T function. Circ Res 2014; 114. doi: 10.1161/CIRCRESAHA.114.302028.
66. Wambach J A, Wegner D J, Yang P, Shinawi M, Baldridge D, Betleja E, et al. Functional characterization of biallelic RTTN variants identified in an infant with microcephaly, simplified gyral pattern, pontocerebellar hypoplasia, and seizures. Pediatr Res 2018; 84:435-441. doi: 10.1038/s41390-018-0083-z.
67. Wang, M. Li, and H. Hakonarson, “ANNOVAR: Functional annotation of genetic variants from high-throughput sequencing data,” Nucleic Acids Res., vol. 38, no. 16, July 2010, doi: 10.1093/nar/gkq603.
68. Wolf, M. J. et al. Drosophila as a model for the identification of genes causing adult human heart disease. Proc. Natl. Acad. Sci. U.S.A 103, 1394-1399 (2006).
69. Woods, L. C., Berbusse, G. W. & Naylor, K. Microtubules are essential for mitochondrial dynamics-fission, fusion, and motility-in Dictyostelium discoideum. Front. Cell Dev. Biol. 4, 19 (2016).
70. Wu M, Kalyanasundaram A, Zhu J. Structural and biomechanical basis of mitochondrial movement in eukaryotic cells. Int J Nanomedicine 2013; 8:4033-4042. doi: 10.2147/IJN.S52132.
71. Zakaria, M. et al. Primary microcephaly, primordial dwarfism, and brachydactyly in adult cases with biallelic skipping of RTTN exon 42. Hum. Mutat. 40, 899-903 (2019).
72. Zebrowski, D. C. et al. Developmental alterations in centrosome integrity contribute to the post-mitotic state of mammalian cardiomyocytes. Elife 4, (2015).

1. A method of treating a cardiomyopathy in a subject in need thereof, comprising administering the subject a Hippo pathway activator compound.

2. A method of claim 1, wherein the Hippo pathway activator compound is selected from Verteporfin, Compound 13, DAY10583, CA3, SBI-115 and C19.

3. A method of claim 2, wherein the Hippo pathway activator compound is C19.

4. A method of claim 1, wherein the subject is a human.

5. A method of claim 1, wherein the subject is a human infant.

6. A method of claim 1, wherein the administration is by intravenous infusion, intravenous injection, subcutaneous injection, or orally.

7. A method of claim 4, wherein the subject suffers from infantile dilated cardiomyopathy, acquired dilated cardiomyopathy, a left ventricular non-compaction cardiomyopathy, a pediatric dilated cardiomyopathy, or heart failure secondary to congenital heart disease.


SEQUENCE LISTING (49 sequences)

SEQ ID NO: 1-2 - skipped (2 entries)

SEQ ID NO: 3 - DNA, 24 bp, Homo sapiens
  misc_feature 1..24: note = father (DNA)
  source 1..24: mol_type = genomic DNA
gttattaaagagttaagcattgcc

SEQ ID NO: 4 - protein, 8 aa, Homo sapiens
  REGION 1..8: note = misc_feature - father (amino acid)
VIKELSIA

SEQ ID NO: 5 - DNA, 24 bp, Homo sapiens
  misc_feature 1..24: note = proband and father (DNA)
  source 1..24: mol_type = genomic DNA
ttcatgggaaaagatgttacaaag

SEQ ID NO: 6 - protein, 8 aa, Homo sapiens
  REGION 1..8: note = misc_feature - proband and father (amino acid)
FMGKDVTK

SEQ ID NO: 7 - DNA, 41 bp, synthetic construct
  misc_feature 1..41: note = Synthetic: sgRNA
  source 1..41: mol_type = other DNA
cgtggaggaaatgctatgtccttcatgggaaaaggtgttac

SEQ ID NO: 8 - DNA, 42 bp, synthetic construct
  misc_feature 1..42: note = Synthetic: FIG. 6C KO-1 - TOP
  source 1..42: mol_type = other DNA
cgtggaggaaatgctatgtcctttcatgggaaaaggtgttac

SEQ ID NO: 9 - DNA, 37 bp, synthetic construct
  misc_feature 1..37: note = Synthetic: FIG. 6C KO-2 - TOP
  source 1..37: mol_type = other DNA
cgtggaggaaatgctatgtcatgggaaaaggtgttac

SEQ ID NO: 10 - DNA, 38 bp, synthetic construct
  misc_feature 1..38: note = Synthetic: FIG. 6C KO-3 - TOP
  source 1..38: mol_type = other DNA
cgtggaggaaatgctatgtccatgggaaaaggtgttac

SEQ ID NO: 11 - DNA, 38 bp, synthetic construct
  misc_feature 1..38: note = Synthetic: FIG. 6C KO-4 - TOP
  source 1..38: mol_type = other DNA
cgtggaggaaatgctatgtccttcagggaaaatgttac

SEQ ID NO: 12 - DNA, 67 bp, Homo sapiens
  variation 7: note = n is g or a
  misc_feature 1..67: note = RTTN
  source 1..67: mol_type = genomic DNA
gaaaagntgttacaaagagcacaattctttgcttgcttcacttatcccatgagatgatggcccaggc

SEQ ID NO: 13 - DNA, 67 bp, synthetic construct
  misc_feature 1..67: note = Synthetic: repair template
  source 1..67: mol_type = other DNA
gaaaaggtgttacaaagagcacaattctttgcttgcttcacttatcccatgagatgatggcccaagc

SEQ ID NO: 14 - DNA, 23 bp, synthetic construct
  misc_feature 1..23: note = Synthetic: primer
  source 1..23: mol_type = other DNA
agcaatgctgtttccttcataag

SEQ ID NO: 15 - DNA, 20 bp, synthetic construct
  misc_feature 1..20: note = Synthetic: primer
  source 1..20: mol_type = other DNA
gagacccaggcaggatctaa

SEQ ID NO: 16 - DNA, 20 bp, synthetic construct
  misc_feature 1..20: note = Synthetic: primer
  source 1..20: mol_type = other DNA
gcaagcttagcactggcaac

SEQ ID NO: 17 - DNA, 23 bp, synthetic construct
  misc_feature 1..23: note = Synthetic: primer
  source 1..23: mol_type = other DNA
ggcaatgttctgtccttcagcag

SEQ ID NO: 18 - DNA, 21 bp, synthetic construct
  misc_feature 1..21: note = Synthetic: primer
  source 1..21: mol_type = other DNA
tgagggattttagcctcttcc

SEQ ID NO: 19 - DNA, 20 bp, synthetic construct
  misc_feature 1..20: note = Synthetic: primer
  source 1..20: mol_type = other DNA
acgaccagggatgagtgttc

SEQ ID NO: 20 - DNA, 23 bp, synthetic construct
  misc_feature 1..23: note = Synthetic: primer
  source 1..23: mol_type = other DNA
caaaaggcaatgtccttcatcag

SEQ ID NO: 21 - DNA, 20 bp, synthetic construct
  misc_feature 1..20: note = Synthetic: primer
  source 1..20: mol_type = other DNA
gtgcaacggcttgtaaatga

SEQ ID NO: 22 - DNA, 20 bp, synthetic construct
  misc_feature 1..20: note = Synthetic: primer
  source 1..20: mol_type = other DNA
tttcatggtcagttggtgga

SEQ ID NO: 23 - DNA, 23 bp, synthetic construct
  misc_feature 1..23: note = Synthetic: primer
  source 1..23: mol_type = other DNA
ggaagccctatatccttcattgg

SEQ ID NO: 24 - DNA, 22 bp, synthetic construct
  misc_feature 1..22: note = Synthetic: primer
  source 1..22: mol_type = other DNA
ggtggaacatgttttccttaaa

SEQ ID NO: 25 - DNA, 21 bp, synthetic construct
  misc_feature 1..21: note = Synthetic: primer
  source 1..21: mol_type = other DNA
ccttcatcccatatgtcccta

SEQ ID NO: 26 - DNA, 23 bp, synthetic construct
  misc_feature 1..23: note = Synthetic: primer
  source 1..23: mol_type = other DNA
atcccaagggaagatggccccag

SEQ ID NO: 27 - DNA, 19 bp, synthetic construct
  misc_feature 1..19: note = Synthetic: primer
  source 1..19: mol_type = other DNA
caggtcccctgtgtctcct

SEQ ID NO: 28 - DNA, 18 bp, synthetic construct
  misc_feature 1..18: note = Synthetic: primer
  source 1..18: mol_type = other DNA
acgttggcaccaggagtg

SEQ ID NO: 29 - DNA, 23 bp, synthetic construct
  misc_feature 1..23: note = Synthetic: primer
  source 1..23: mol_type = other DNA
ctcccaggaaatgatggccagag

SEQ ID NO: 30 - DNA, 20 bp, synthetic construct
  misc_feature 1..20: note = Synthetic: primer
  source 1..20: mol_type = other DNA
ggaaatgcccagtgtctgtt

SEQ ID NO: 31 - DNA, 19 bp, synthetic construct
  misc_feature 1..19: note = Synthetic: primer
  source 1..19: mol_type = other DNA
agtctgaccctgcccactc

SEQ ID NO: 32 - DNA, 23 bp, synthetic construct
  misc_feature 1..23: note = Synthetic: primer
  source 1..23: mol_type = other DNA
ctcccaggaaatgatggccagag

SEQ ID NO: 33 - DNA, 20 bp, synthetic construct
  misc_feature 1..20: note = Synthetic: primer
  source 1..20: mol_type = other DNA
gcttgttggctccagtcttc

SEQ ID NO: 34 - DNA, 20 bp, synthetic construct
  misc_feature 1..20: note = Synthetic: primer
  source 1..20: mol_type = other DNA
gccccagtggtcatacattt

SEQ ID NO: 35 - DNA, 23 bp, synthetic construct
  misc_feature 1..23: note = Synthetic: primer
  source 1..23: mol_type = other DNA
ctcccaggaaatgatggccagag

SEQ ID NO: 36 - DNA, 22 bp, synthetic construct
  misc_feature 1..22: note = Synthetic: primer
  source 1..22: mol_type = other DNA
tggtctgtttttgctgattctt

SEQ ID NO: 37 - DNA, 19 bp, synthetic construct
  misc_feature 1..19: note = Synthetic: primer
  source 1..19: mol_type = other DNA
ccgggaggctttctagcat

SEQ ID NO: 38 - DNA, 23 bp, synthetic construct
  misc_feature 1..23: note = Synthetic: primer
  source 1..23: mol_type = other DNA
gtcacatgtgaggatggcccagg

SEQ ID NO: 39 - DNA, 20 bp, synthetic construct
  misc_feature 1..20: note = Synthetic: primer
  source 1..20: mol_type = other DNA
ccccgacatgactctttgaa

SEQ ID NO: 40 - DNA, 20 bp, synthetic construct
  misc_feature 1..20: note = Synthetic: primer
  source 1..20: mol_type = other DNA
tctcctcccaagtccttgtc

SEQ ID NO: 41 - DNA, 25 bp, synthetic construct
  misc_feature 1..25: note = Synthetic: primer
  source 1..25: mol_type = other DNA
tctttatgagtgacgataactccat

SEQ ID NO: 42 - DNA, 20 bp, synthetic construct
  misc_feature 1..20: note = Synthetic: primer
  source 1..20: mol_type = other DNA
atatttcattatcctacaag

SEQ ID NO: 43 - DNA, 20 bp, synthetic construct
  misc_feature 1..20: note = Synthetic: primer
  source 1..20: mol_type = other DNA
ttttccggaggttccaatgc

SEQ ID NO: 44 - DNA, 24 bp, Homo sapiens
  misc_feature 1..24: note = mother (DNA)
  source 1..24: mol_type = genomic DNA
ttcatgggaaaaggtgttacaaag

SEQ ID NO: 45 - protein, 8 aa, Homo sapiens
  REGION 1..8: note = misc_feature - mother (amino acid)
FMGKGVTK

SEQ ID NO: 46 - DNA, 31 bp, synthetic construct
  misc_feature 1..31: note = Synthetic: FIG. 6C KO-1 - BOTTOM
  source 1..31: mol_type = other DNA
cgtggaggaaatgctatggaaaaggtgttac

SEQ ID NO: 47 - DNA, 35 bp, synthetic construct
  misc_feature 1..35: note = Synthetic: FIG. 6C KO-2 - BOTTOM
  source 1..35: mol_type = other DNA
cgtggaggaaatgctatgtccttaaaaggtgttac

SEQ ID NO: 48 - DNA, 36 bp, synthetic construct
  misc_feature 1..36: note = Synthetic: FIG. 6C KO-3 - BOTTOM
  source 1..36: mol_type = other DNA
cgtggaggaaatgctatgcatgggaaaaggtgttac

SEQ ID NO: 49 - DNA, 36 bp, synthetic construct
  misc_feature 1..36: note = Synthetic: FIG. 6C KO-4 - BOTTOM
  source 1..36: mol_type = other DNA
cgtggaggaaatgctatgtccgggaaaaggtgttac